(19) United States
(12) Patent Application Publication
(10) Pub. No.: US 2025/0102523 A1
(43) Pub. Date: Mar. 27, 2025

(54) BIOMARKERS FOR DETECTING SYNOVIAL JOINT DISEASE IN MUCOPOLYSACCHARIDOSES

(71) Applicant: THE TRUSTEES OF THE UNIVERSITY OF PENNSYLVANIA, Philadelphia, PA (US)

(72) Inventors: Lachlan James SMITH, Philadelphia, PA (US); Chenghao ZHANG, Newton Highlands, MA (US)

(21) Appl. No.: 18/832,637

(22) PCT Filed: Jan. 26, 2023
(86) PCT No.: PCT/US2023/061389

§ 371 Date: Jul. 24, 2024

Related U.S. Application Data
(60) Provisional application No. 63/303,403, filed on Jan. 26, 2022.
(60) Provisional application No. 63/436,664, filed on Jan. 2, 2023.
(60) Provisional application No. 63/478,311, filed on Jan. 3, 2023.

(51) Int. Cl. G01N 33/68

(52) U.S. Cl. CPC G01N 33/6893; G01N 2800/042; G01N 2800/52

(57) ABSTRACT

Disclosed are methods for detecting synovial joint disease in mucopolysaccharidoses. The methods use a panel of serum biomarker proteins and measure a difference in abundance of one or more of the biomarkers between a subject and a control to identify and treat synovial joint disease in mucopolysaccharidoses. The methods allow for early detection and early therapeutic treatment of synovial joint disease in mucopolysaccharidoses to stop progression of the disease.

### GOVERNMENT RIGHTS

This invention was made with government support under AR071975 awarded by the National Institutes of Health. The government has certain rights in the invention.

### TECHNICAL FIELD

Disclosed herein are methods for detecting synovial joint disease in mucopolysaccharidoses.

### BACKGROUND

The mucopolysaccharidoses (MPS) are a family of inherited lysosomal storage diseases characterized by deficient activity of enzymes that degrade glycosaminoglycans (GAGs) due to mutations in associated genes [1]. There are 11 subtypes of MPS, each exhibiting a mutation (and resulting deficient activity) in a different lysosomal enzyme [1]. Disease prevalence across all subtypes is approximately 1 in 25,000 births (NINDS). MPS children display various symptoms depending on disease subtype and severity, but commonly present with a mix of one or more of the following phenotypes: hepatosplenomegaly, corneal clouding, upper airway disease, cardiac defects, progressive CNS deterioration, abnormal facies, skeletal dysplasia (dysostosis multiplex) and varying degrees of short stature [1]. Synovial joint (e.g. hip, knee, hands and shoulder) abnormalities in MPS are prevalent, and patients experience significantly decreased quality of life due to pain and mobility impairment [2]. Studies suggest that progressive joint disease can be traced to a combination of developmental abnormalities and chronic inflammation, which accelerates soft tissue degeneration [3, 4].

There remains a need in the clinic for specific biomarkers for assessment of joint disease progression and response to therapy. The present disclosure addresses these needs.

### SUMMARY

In meeting these needs, the present disclosure provides methods for detecting synovial joint disease in mucopolysaccharidoses (MPS). The methods can include measuring a level of at least one protein in a sample from a subject and comparing it to that in a sample from a control subject. The methods can include measuring levels of at least one, at least two, at least three, at least four, at least five, at least six, at least seven, at least eight, at least nine, or at least ten proteins in a sample from a subject and comparing these levels to those in a sample from a control subject. The one or more proteins can be selected from the panel of proteins presented in Table 2. The one or more proteins can comprise matrix metalloproteinase 19 (MMP19), inter-alpha-trypsin inhibitor heavy chain 3 (ITIH3), chemerin (RARRES2), C-reactive protein (CRP), alpha-mannosidase (MAN2B1), cluster differentiation (CD)86, alpha-1-microglobulin (AMBP), prosaposin (PSAP), secreted phosphoprotein 2 (SPP2), and V-set and immunoglobulin domain containing 4 (VSIG4).

The levels of the one or more proteins can have a log 2 fold change between about −2.5 and about 5 relative to the levels of these proteins in a sample from a control subject. The levels of the one or more proteins can have between about 0.2- and about 30-fold difference relative to the levels of these proteins in a sample from a control subject.

The methods also include administering to the subject having a log 2 fold change between about −2.5 and about 5 in the level(s) of the one or more protein(s) relative to the level(s) of the protein(s) in a sample from a control subject an MPS treatment. The methods can include administering to the subject having between about 0.2- and about 30-fold difference in the level(s) of the one or more protein(s) relative to the level(s) of these protein(s) in a sample from a control subject an MPS treatment. Suitable MPS treatments include enzyme replacement therapy, bone marrow transplantation, surgery, and physical therapy. The step of administering the therapy can be earlier in the life of the subject when the level of the one or more proteins is measured and a fold change difference in the level(s) of the one or more proteins is detected than without this measuring step.

Exhibit A attached hereto is incorporated herein by reference in its entirety for any and all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The summary, as well as the following detailed description, is further understood when read in conjunction with the appended drawings. For the purpose of illustrating the disclosed compositions and methods there are shown in the drawings exemplary embodiments of compositions and methods; however, these should not be limited to the specific embodiments disclosed. In the drawings:

FIGS. 1A and 1B are principal component analysis plots showing clustering of samples as a function of disease state for both synovial fluid (FIG. 1A) and serum (FIG. 1B). FIGS. 1C and 1D are heat maps showing differences in protein abundance between MPS I and heterozygous control samples for synovial fluid (FIG. 1C) and serum (FIG. 1D).

FIG. 2 is a bar graph showing candidate protein biomarkers that exhibit significantly elevated abundance in both synovial fluid and serum from MPS I dos compared to heterozygous controls (all p<0.05).

FIGS. 3A-3D are plots for candidate biomarkers showing strong correlations between protein abundance in synovial fluid and serum. R=Spearman correlation coefficient.

FIGS. 4A-4D are images of representative immunohistochemical staining of ITIH3 in the knee joint articular cartilage of MPS I dogs at 12 months of age. MPS I dogs exhibit markedly elevated ITIH3 expression by superficial zone chondrocytes compared to those in controls. Left: low magnification view (scale=100 μm); right: higher magnification view of the region indicated (scale=30 μm).

FIGS. 5A-5F are bar graphs showing tensile mechanical properties of anterior cruciate ligaments from control (n=4, left bars) and MPS I (n=5, right bars) dogs at 12 months-of-age. *p<0.05 vs control.

FIGS. 6A-6J are graphs showing correlations between candidate serum biomarker absorbance and anterior cruciate ligament (ACL) stiffness; r=Spearman correlation coefficients.

FIGS. 7A-7J are graphs showing correlations between candidate serum biomarker absorbance and ACL toughness; r=Spearman correlation coefficients.

Exhibit A FIGS. 8A-13N:

FIGS. 8A-8F. Global analysis of proteomic screening of synovial fluid and serum from 12-month-old MPS I (n=6) and healthy control (n=5) dogs. A-B. Principal component analyses; C-D. Heat maps; and E-F. Volcano plots, showing clustering of samples, and relative up and down regulation of proteins as a function of disease state.

FIGS. 9A-9E. Up and down regulation of selected proteins with significantly different abundance in MPS I dogs compared to controls. A. Top 10 proteins significantly upregulated and B. Downregulated in MPS I synovial fluid. C. Top 10 proteins significantly upregulated and D. Downregulated in MPS I serum. E. Selected proteins significantly up or down regulated in both MPS I synovial fluid and serum with previously described roles in inflammatory joint disease or lysosomal dysfunction. All p<0.05, N=5-6.

FIGS. 10-10D. Pathway analysis of proteomic screening results for synovial fluid and serum from 12-month-old MPS I (n=6) and healthy control (n=5) dogs. A-B. Gene ontology (GO) analysis. C-D. Kyoto Encyclopedia of Genes and Genomes (KEGG) pathway enrichment analysis.

FIGS. 11A-11F. Representative immunostaining and quantification of cells immunopositive for A-B. ITIH3; C-D. MMP19; and E-F. A1M in the femoral condylar cartilage of control and MPS I animals at 12 months-of-age. Scale=100 μm (higher magnification=30 μm); *p<0.05 vs control; N=5-6; mean±SD; unpaired t-test; N=5-6.

FIGS. 12A-12T. Magnetic resonance imaging (MRI) of stifle joints from 12-month-old control and MPS I dogs. A. Representative T2-weighted, mid-sagittal images showing fluid effusions cranial (yellow arrow) and caudal (teal arrow) to the joint in an MPS I dog. B. Representative proton density-weighted, dorsal plane images showing meniscal intrasubstance degeneration (red arrow) in an MPS I dog. C-L. Semi-quantitative MRI grades. *p<0.05 vs control; median and interquartile range; Mann-Whitney Test; N=5-6. M-P. Spearman correlations between protein abundance (normalized log 2 intensity) in synovial fluid and overall MRI grade. Q-T. Spearman correlations between protein abundance (normalized log 2 intensity) in serum and overall MRI grade.

FIGS. 13A-13N. Histological assessment of femoral condylar cartilage. A. Representative mid-sagittal sections of femoral medial condylar cartilage from control and MPS I animals at 12 months-of-age. Scale=2 mm; Safranin O/fast green staining. B. Higher magnification views of the regions indicated in A. showing cell clustering, enlargement and increased density in MPS I cartilage. Scale=100 μm (inset 30 μm). Quantification of: C. Chondrocyte pathology; D. Proteoglycan staining; E. Cartilage structure; and F. Overall cartilage grade. *p<0.05 vs control; median and interquartile range; Mann-Whitney Test; N=5-6. G-J. Spearman correlations between protein abundance (normalized log 2 intensity) in synovial fluid and overall cartilage grade. K-N. Spearman correlations between protein abundance (normalized log 2 intensity) in serum and overall cartilage grade.

### DETAILED DESCRIPTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein. Any one or more parts of any described aspect can be combined with any one or more parts of any other described aspect or described aspects.

The term “substantial” refers to a degree of similarity, difference, increase, or decrease, as in a comparison to a known value. Substantial can include at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% similarity, difference, increase, or decrease, as in a comparison to a known value.

The terms “treating” or “treatment” refer to any success or indicia of success in the attenuation or amelioration of joint pain, including any objective or subjective parameter such as abatement, diminishing of one or more symptoms of joint pain, or making the injury, pathology, or condition more tolerable to the subject, improving the subject's physical well-being, or prolonging the length of survival. The treatment or amelioration of the one or more symptoms can be based on objective or subjective parameters; including the results of a physical examination, laboratory test(s), non-invasive imaging test(s), and/or self-reporting by the subject.

The term “subject” as used herein is intended to mean a mammal. The methods described herein are applicable to human and nonhuman animals, although preferably used with pets and humans, and most preferably with humans. “Subject” and “patient” are used interchangeably herein. In some embodiments, the subject is human.

The term “control subject” as used herein is intended to mean a subject who is healthy, who does not suffer from mucopolysaccharidoses (MPS).

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. The materials, methods, and examples disclosed herein are illustrative only and not intended to be limiting.

It is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise. The term “about” as used herein when referring to a measurable value such as an amount, a temporal duration, and the like, is meant to encompass variations of ±10%, ±5%, ±1%, or ±0.1% from the specified value, as such variations are appropriate to perform the disclosed methods.

As used herein, approximating language may be applied to modify any quantitative representation that may vary without resulting in a change in the basic function to which it is related. Accordingly, a value modified by “about” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only.

As used in this specification and the appended claims, the singular forms “a,” “an,” and “the” include plural referents unless the content clearly dictates otherwise. Thus, for example, reference to “a cell” includes a combination of two or more cells, and the like.

### Methods of Detecting Synovial Joint Disease

The disclosed methods can comprise measuring a level of at least one protein in a sample from a subject, the at least one protein selected from the panel of proteins as presented in Table 2, where the level of the at least one protein has a log 2 fold change between about −2.5 and about 5 relative to the level of the at least one protein in a sample from a control subject. The disclosed methods can further comprise administering to the subject a mucopolysaccharidoses (MPS) treatment after the measuring step. The MPS treatments can include one or more of an enzyme replacement therapy, bone marrow transplantation, surgery, and physical therapy.

The methods may include measuring the levels of more than one protein. In some embodiments, the level(s) of the protein(s) in the subject can have a log 2 fold change between about −2.5 and about 5 relative to the level(s) of the corresponding protein(s) in a sample from a control subject. Suitable log 2 fold change values include between about −2.5 and about 5, between about −2.2 and about 5, between about −2 and about 5, between about −1.8 and about 5, between about −1.6 and about 5, between about −1.4 and about 5, between about −1.2 and about 5, between about −1 and about 5, between about −0.8 and about 5, between about −0.6 and about 5, between about −0.4 and about 5, between about −0.2 and about 5, between about 0.2 and about 5, between about 0.4 and about 5, between about 0.6 and about 5, between about 0.8 and about 5, between about 1 and about 5, between about 1.2 and about 5, between about 1.4 and about 5, between about 1.6 and about 5, between about 1.8 and about 5, between about 2 and about 5, between about 2.2 and about 5, between about 2.4 and about 5, between about 2.6 and about 5, between about 2.8 and about 5, and between about 3 and about 5.

In some aspects, suitable log 2 fold change values include between about −2.5 and about 3, between about −2.2 and about 3, between about −2 and about 3, between about −1.8 and about 3, between about −1.6 and about 3, between about −1.4 and about 3, between about −1.2 and about 3, between about −1 and about 3, between about −0.8 and about 3, between about −0.6 and about 3, between about −0.4 and about 3, between about −0.2 and about 3, between about 0.2 and about 3, between about 0.4 and about 3, between about 0.6 and about 3, between about 0.8 and about 3, between about 1 and about 3, between about 1.2 and about 3, between about 1.4 and about 3, between about 1.6 and about 3, between about 1.8 and about 3, between about 2 and about 3, between about 2.2 and about 3, between about 2.4 and about 3, between about 2.6 and about 3, and between about 2.8 and about 3.

In some aspects, the disclosed methods can comprise measuring a level of at least one protein in a sample from a subject, the at least one protein selected from the panel of proteins as presented in Table 2, where the level of the at least one protein has between about 0.2- and about 30-fold difference relative to the level of the at least one protein in a sample from a control subject.

The methods may include measuring the levels of more than one protein. In some embodiments, the level(s) of the protein(s) in the subject can have fold difference values between about 0.2 and about 30 relative to the level(s) of the corresponding protein(s) in a sample from a control subject. Suitable fold difference values include between about 0.2 and about 30, between about 0.4 and about 30, between about 0.6 and about 30, between about 0.8 and about 30, between about 1.2 and about 30, between about 1.4 and about 30, between about 1.6 and about 30, between about 1.8 and about 30, between about 2 and about 30, between about 2.2 and about 30, between about 2.4 and about 30, between about 2.6 and about 30, between about 2.8 and about 30, between about 3 and about 30, between about 3.2 and about 30, between about 3.4 and about 30, between about 3.6 and about 30, between about 3.8 and about 30, between about 4 and about 30, between about 5 and about 30, between about 6 and about 30, between about 7 and about 30, between about 8 and about 30, between about 9 and about 30, between about 10 and about 30, between about 15 and about 30, between about 20 and about 30, and between about 25 and about 30.

In some aspects, suitable fold difference values include between about 0.2 and about 10, between about 0.4 and about 10, between about 0.6 and about 10, between about 0.8 and about 10, between about 1.2 and about 10, between about 1.4 and about 10, between about 1.6 and about 10, between about 1.8 and about 10, between about 2 and about 10, between about 2.2 and about 10, between about 2.4 and about 10, between about 2.6 and about 10, between about 2.8 and about 10, between about 3 and about 10, between about 3.2 and about 10, between about 3.4 and about 10, between about 3.6 and about 10, between about 3.8 and about 10, between about 4 and about 10, between about 5 and about 10, between about 6 and about 10, between about 7 and about 10, between about 8 and about 10, and between about 9 and about 10.

The disclosed methods can include measuring the level(s) of at least one, at least two, at least three, at least four, at least five, at least six, at least seven, at least eight, at least nine, or at least ten proteins selected from the panel presented in Table 2. In some aspects, the at least one, the at least two, the at least three, the at least four, the at least five, the at least six, the at least seven, the at least eight, the at least nine, or the at least ten proteins comprise matrix metalloproteinase 19 (MMP19), inter-alpha-trypsin inhibitor heavy chain 3 (ITIH3), chemerin (RARRES2), C-reactive protein (CRP), alpha-mannosidase (MAN2B1), cluster differentiation (CD)86, alpha-1-microglobulin (AMBP), prosaposin (PSAP), secreted phosphoprotein 2 (SPP2), and V-set and immunoglobulin domain containing 4 (VSIG4).

The measuring can include measuring in a sample obtained from a subject and/or in a sample obtained from a control subject. The sample can be any one or more of synovial fluid, blood, serum, saliva, urine, and pleural fluid. In some aspects, the sample is serum. In some aspects, the sample is synovial fluid treated with hyaluronidase. In some aspects, the sample is blood. In some aspects, the sample is saliva. In some aspects, the sample is urine. In some aspects, the sample is pleural fluid.

In some aspects, the measuring of the protein levels comprises measuring by a suitable method, such as by Western blotting, enzyme-linked immunosorbent assay (ELISA), multiplex ELISA, and/or high performance liquid chromatography (HPLC). In some embodiments, measuring is by Western blotting. In some embodiments, measuring is by ELISA. In some embodiments, measuring is by multiplex ELISA. In some embodiments, measuring is by HPLC.

The subject and the control subject can be mammals. The subject and the control subject can be humans. In some aspects, the subject and the control subject are children. In some aspects, the subject and the control subject are adolescents.

In some aspects, the subject has diminished biomechanical properties of anterior cruciate ligament (ACL). The diminished biomechanical properties of ACL in the subject can include any one or more of reduced stiffness (N/mm), reduced modulus (MPa), reduced toughness (J·m⁻³), reduced failure load (N), reduced failure stress (N/mm²), and reduced failure strain (mm/mm) as compared to those in the control subject and as measured using servo-hydraulic mechanical testing system. In some embodiments, the diminished biomechanical properties of ACL in the subject is reduced ACL stiffness (N/mm), as compared to that in the control subject and as measured using servo-hydraulic mechanical testing system. In some embodiments, the diminished biomechanical properties of ACL in the subject is reduced ACL toughness (J·m⁻³), as compared to that in the control subject and as measured using servo-hydraulic mechanical testing system.

In some embodiments, the diminished biomechanical properties of ACL are detected in the subject when the serum protein level(s) of the one or more proteins selected from the panel of proteins as presented in Table 2 have a log 2 fold change between about −2.5 and about 5 relative to the level(s) of the corresponding protein(s) in the serum of the control subject. For example, any one of the diminished biomechanical properties of ACL can be detected in the subject when the serum protein level(s) of the one or more proteins selected from the panel of proteins as presented in Table 2 has log 2 fold change values between about −2.5 and about 5, between about −2.2 and about 5, between about −2 and about 5, between about −1.8 and about 5, between about −1.6 and about 5, between about −1.4 and about 5, between about −1.2 and about 5, between about −1 and about 5, between about −0.8 and about 5, between about −0.6 and about 5, between about −0.4 and about 5, between about −0.2 and about 5, between about 0.2 and about 5, between about 0.4 and about 5, between about 0.6 and about 5, between about 0.8 and about 5, between about 1 and about 5, between about 1.2 and about 5, between about 1.4 and about 5, between about 1.6 and about 5, between about 1.8 and about 5, between about 2 and about 5, between about 2.2 and about 5, between about 2.4 and about 5, between about 2.6 and about 5, between about 2.8 and about 5, or between about 3 and about 5 relative to the level(s) of the one or more proteins in a sample from a control subject.

In some embodiments, the diminished biomechanical properties of ACL are detected in the subject when the serum protein level(s) of the one or more proteins selected from the panel of proteins as presented in Table 2 has between about 0.2- and about 30-fold difference relative to the level(s) of the corresponding protein(s) in the serum of the control subject. For example, any one of the diminished biomechanical properties of ACL can be detected in the subject when the serum protein level(s) of the one or more proteins selected from the panel of proteins as presented in Table 2 has fold difference between about 0.2 and about 30, between about 0.4 and about 30, between about 0.6 and about 30, between about 0.8 and about 30, between about 1.2 and about 30, between about 1.4 and about 30, between about 1.6 and about 30, between about 1.8 and about 30, between about 2 and about 30, between about 2.2 and about 30, between about 2.4 and about 30, between about 2.6 and about 30, between about 2.8 and about 30, between about 3 and about 30, between about 3.2 and about 30, between about 3.4 and about 30, between about 3.6 and about 30, between about 3.8 and about 30, between about 4 and about 30, between about 5 and about 30, between about 6 and about 30, between about 7 and about 30, between about 8 and about 30, between about 9 and about 30, between about 10 and about 30, between about 15 and about 30, between about 20 and about 30, or between about 25 and about 30 relative to the level(s) of the one or more proteins in a sample from a control subject.

In the disclosed methods, the step of administering the treatment to the subject can occur earlier in the life of the subject with the measuring step than without the measuring step. For example, the subject may be administered the treatment at least two months, at least four months, at least six months, at least eight months, at least ten months, at least one year, at least one and a half years, at least two years, at least two and a half years, at least three years, at least three and a half years, at least four year, at least four and a half years, at least five years, earlier when the serum protein level(s) of the one or more proteins selected from the panel of proteins as presented in Table 2 is measured according to the disclosed methods than without the measuring step.

The treatment can include an MPS treatment, such as enzyme replacement therapy, bone marrow transplantation, surgery, and physical therapy. The treatment can include intra-articular enzyme administration, gene therapy, and anti-inflammatory therapy. The anti-inflammatory treatments can include administering to the subject non-steroidal anti-inflammatory medications, disease-modifying antirheumatic drugs (DMARDs), steroids, and anti-tumor necrosis factor (TNF)-α therapies.

### EXAMPLES

### Example 1. Proteomic Screening Identifies Novel Biomarkers of Synovial Joint Disease in Mucopolysaccharidosis I Dogs

MPS I is characterized by deficient α-L-iduronidase (IDUA) activity, leading to progressive accumulation of poorly degraded heparan and dermatan sulfate GAGs in cells and tissues [2]. The naturally-occurring canine model of MPS I exhibits progressive synovial joint abnormalities similar to human patients, making it a clinically-relevant platform for biomarker discovery. The objectives of this study was to undertake an unbiased proteomic screen to identify molecular biomarkers upregulated in the synovial fluid (SF) of MPS I dogs, and to identify circulating (serum) biomarker candidates that may serve as strong predictors of synovial joint disease.

### Materials and Methods

Animals and Sample Collection: With IACUC approval, serum and SF was collected from 6×MPS I affected and 5× heterozygous control dogs at 12 months-of-age. Blood was collected from the cephalic vein, allowed to clot, then centrifuged and serum collected. Animals were then euthanized, stifle (knee) joints opened, and SF collected (about 200 μl) using an 18-gauge needle.

Mass Spectrometry: Total protein concentration was assessed using the bicinchoninic acid assay. SF was pretreated with hyaluronidase, and proteins in both SF and serum were denatured, reduced, alkylated, and digested into peptides. Peptide separation and mass spectrometric analyses were carried out using a Thermo Scientific UltiMate 3000 UPLC coupled to a Q Exactive HF Orbitrap LC-MS/MS System.

Bioinformatics and Statistical Analysis: Peptides were identified using Spectronaut software and Perseus was used to establish statistically-significant fold changes in protein abundance in MPS I vs control samples. Spearman's rank-order tests were used to examine correlations between serum and synovial fluid (p<0.05).

### Results

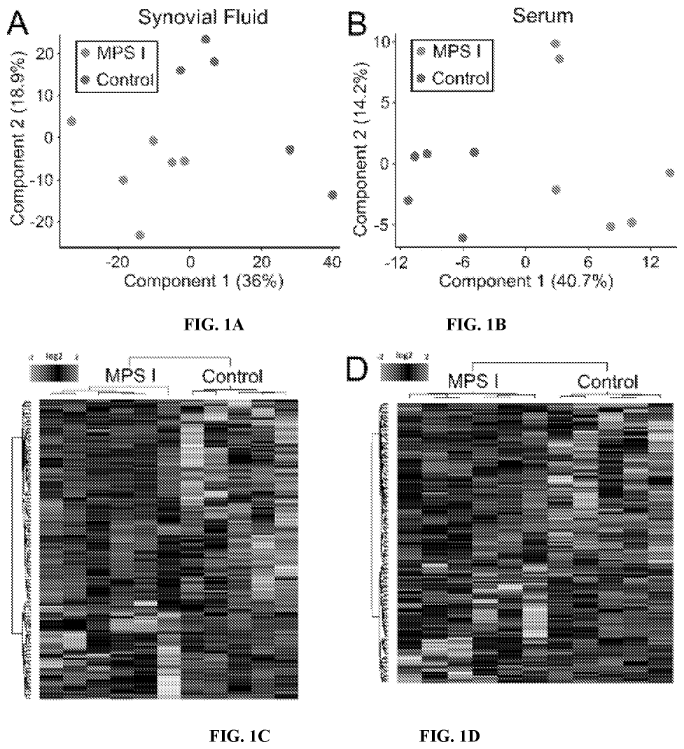

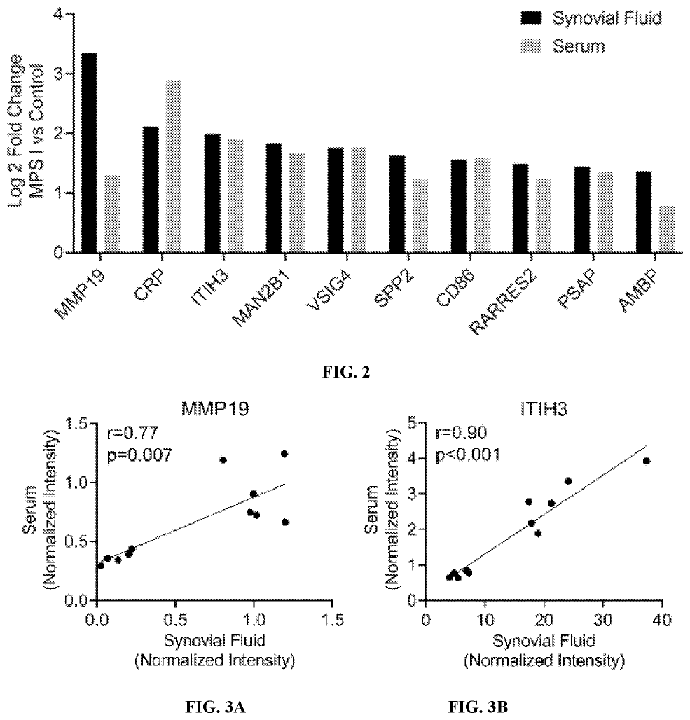

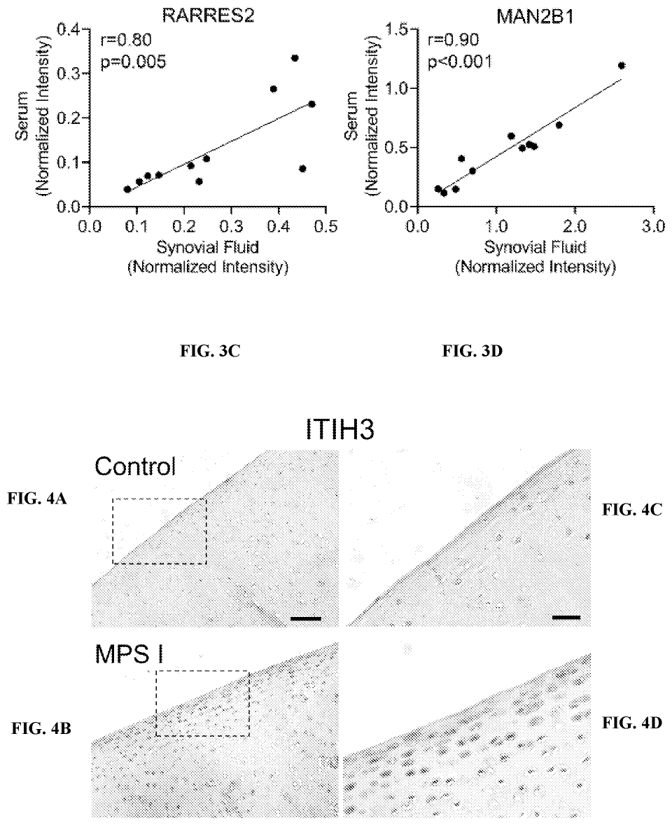

In SF and serum samples, mass spectrometry identified 812 and 415 unique proteins, respectively. Principal component analyses (FIGS. 1A and B) demonstrated clustering of control and MPS I samples for both SF and serum, confirming significant effects of disease state on relative protein abundance. Examining SF, there were 151 proteins that exhibited significantly different abundance in MPS I vs control (FIG. 1C), and of these, 104 exhibited a log 2 fold change >1. For serum, there were 154 proteins that exhibited significantly different abundance in MPS I vs control (FIG. 1D), and of these, 64 exhibited a log 2 fold change >1. There were 50 proteins for which abundance was significantly different for both SF and serum, and of those, 40 exhibited a significant correlation in abundance between SF and serum across all 11 samples (Table 2). To identify a shortlist of 10 biomarker candidates predictive of joint disease, these proteins were ranked according to their relative elevation in SF (MPS I vs control, FIG. 2). Spearman correlation coefficients (SF vs serum) ranged from 0.64 to 0.90 for these 10 proteins (FIGS. 3A-3D and Table 1), showing that abundance in serum was highly predictive of abundance in synovial fluid.

TABLE 1
Receiver operator characteristic (SF) and correlation (SF
vs serum) analysis results for candidate biomarkers.

| | |
|---|---|
| ROC (SF) | Spearman (SF vs Serum) |

| Molecule | AUC | p value | r | p value |
|---|---|---|---|---|
| MMP19 | 1.000 | 0.0062 | 0.7727 | 0.0074 |
| CRP | 0.9333 | 0.0176 | 0.8818 | 0.0007 |
| ITIH3 | 1.000 | 0.0062 | 0.9 | 0.0004 |
| MAN2B1 | 1.000 | 0.0062 | 0.9 | 0.0004 |
| VSIG4 | 1.000 | 0.0062 | 0.6818 | 0.0251 |
| SPP2 | 1.000 | 0.0062 | 0.8636 | 0.0012 |
| CD86 | 1.000 | 0.0062 | 0.6364 | 0.0402 |
| RARRES2 | 1.000 | 0.0062 | 0.8 | 0.0047 |
| PSAP | 1.000 | 0.0062 | 0.8424 | 0.0037 |
| AMBP | 1.000 | 0.0062 | 0.8727 | 0.0009 |

In this study, novel candidate biomarkers of synovial joint disease in MPS I were identified using the clinically-relevant canine model. Importantly, there were strong correlations between the abundance of these biomarkers in SF and serum, showing that they serve as circulating biomarkers that specifically reflect joint disease severity and enhancing their clinical utility. These markers also provide novel insights into mechanisms of joint disease. For example, MMP19 (matrix metalloproteinase-19) cleaves aggrecan, a major component of healthy articular cartilage [5]. ITIH3 (inter-α-trypsin inhibitor heavy chain 3), while known to play a role in several neurological diseases, is also important for matrix stabilization [6]. RARRES2 (retinoic acid receptor responder 2 or chemerin) is an adipokine and inflammatory mediator that is elevated in both osteoarthritis and rheumatoid arthritis [7, 8]. Finally, MAN2B1 (alpha-mannosidase) is a lysosomal hydrolyze, and its elevated abundance reflects broader lysosomal dysfunction secondary to IDUA deficiency.

Previous work by other labs has taken a much narrower approach to biomarker discovery, rather than conducting an unbiased screen as we have performed. These prior studies have predominantly focused on inflammatory mediators and extracellular matrix breakdown products. For example, a recent study (Lund et al., JIMD Reports, 2021; 58:89-99) showed that the inflammatory cytokine IL-6, and the collagen breakdown product pyridinoline measured in plasma were predictive of joint dysfunction. These molecules have been widely studied in other inflammatory and degenerative joint diseases (such as arthritis) and may not be sensitive and specific for joint disease associated with MPS.

### Example 2. Immunohistochemical Validation of Local Biomarker Expression in MPS I Dog Joints

Immunohistochemistry was used to confirm that cells present in joint tissues are a source of biomarkers detected in synovial fluid and serum.

### Materials and Methods

Specifically, distal femurs from 12 month-old MPS I (n=6) and healthy control (n=5) dogs were fixed, decalcified and processed for paraffin histology. The histology used primary anti-ITIH3 antibody Cat #: ab197188 (Abcam®) and the detection was carried out using a kit, cat #PK-6200 (VectorLabs) that included a secondary biotinylated horse anti-mouse/rabbit IgG antibody. The substrate used was 3,3′Diaminobenzidine.

### Results

Proteomics identified Inter-Alpha-Trypsin Inhibitor Heavy Chain 3 (ITIH3) as exhibiting significantly elevated expression in both the synovial fluid and serum of MPS I dogs, and expression was found to be highly correlated in serum and synovial fluid across our study cohort (r=0.9).

While ITIH3 is known to play a role in several neurological diseases, and is also thought to be important for matrix stabilization, virtually nothing is known about its role in joint health.

Chondrocytes in the superficial zone of MPS I knee joint articular cartilage exhibited markedly elevated expression of ITIH3 compared to healthy control chondrocytes (FIGS. 4A-4D). This finding strongly supports ITIH3 as a specific biomarker of joint and cartilage disease in MPS I, as a molecule that is expressed locally by joint tissues, and which is also measurable systemically in serum.

### Example 3. Serum Biomarkers Predict Abnormal MPS I Dog Joint Biomechanical Function

### Materials and Methods

Anterior cruciate ligaments (ACLs) were isolated from biomarker donor-matched MPS I (n=5) and healthy control (n=4) dogs and tested in uniaxial tension to failure using a servo-hydraulic mechanical testing system (Model 8874; Instron; Norwood, MA, USA). The ACL is critical to the stability and biomechanical functioning of the knee joint.

Left stifle (knee) joints were excised postmortem, all soft tissue between the distal femur and proximal tibia except the ACL was carefully removed, and ACL cross-sectional area was measured using a laser-based device. The femur and tibia were potted in polymethylmethacrylate, mounted in custom fixtures of a servohydraulic mechanical testing system (Instron 8874), and the ACL tested in uniaxial tension to failure. Briefly, following 10 cycles of preconditioning, samples were subjected to a quasi-static ramp to failure at a strain rate of 0.3%/second. Testing was conducted at room temperature and samples were sprayed with saline to prevent dehydration. The following parameters were calculated for all samples: stiffness, modulus, toughness, failure load, failure stress and failure strain. Significant differences in ACL tensile mechanical properties between MPS I and controls were established using Mann-Whitney tests (p<0.05).

### Results

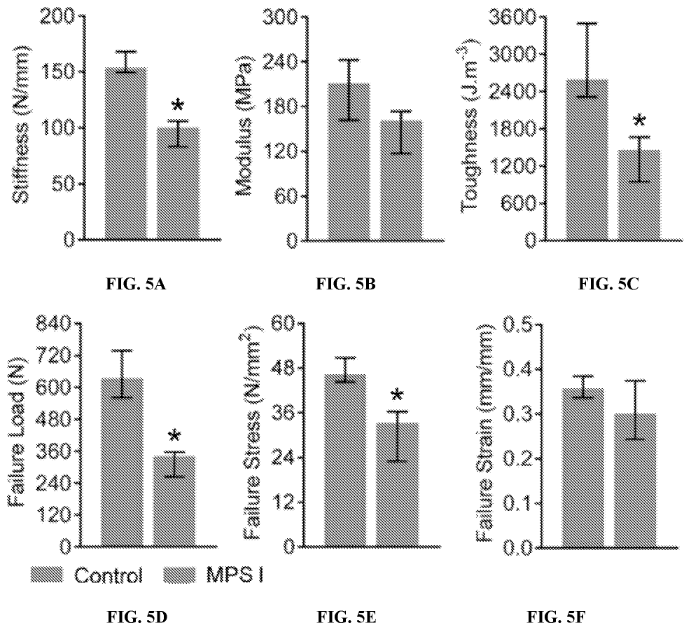

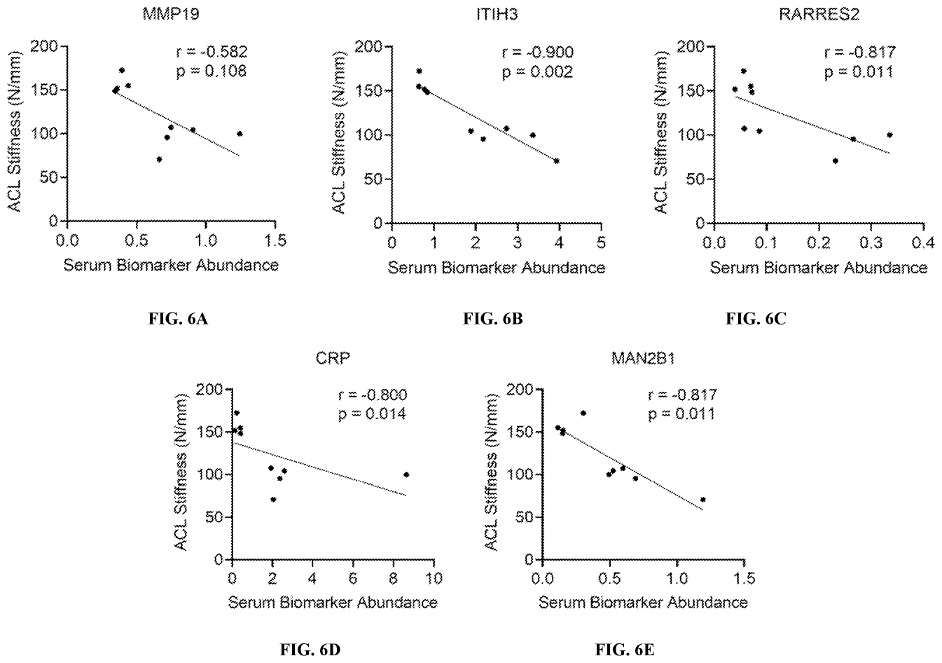

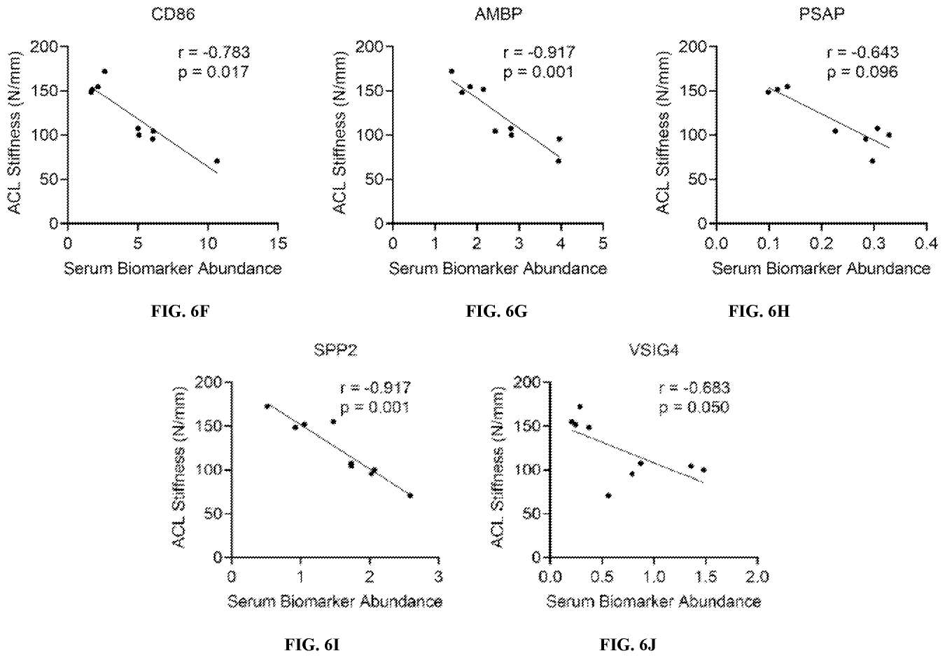

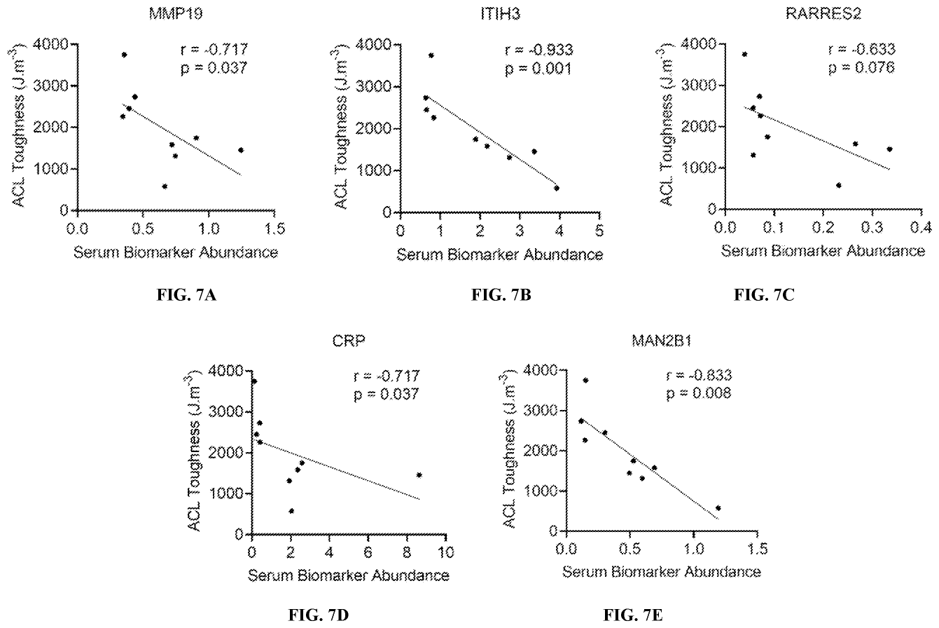

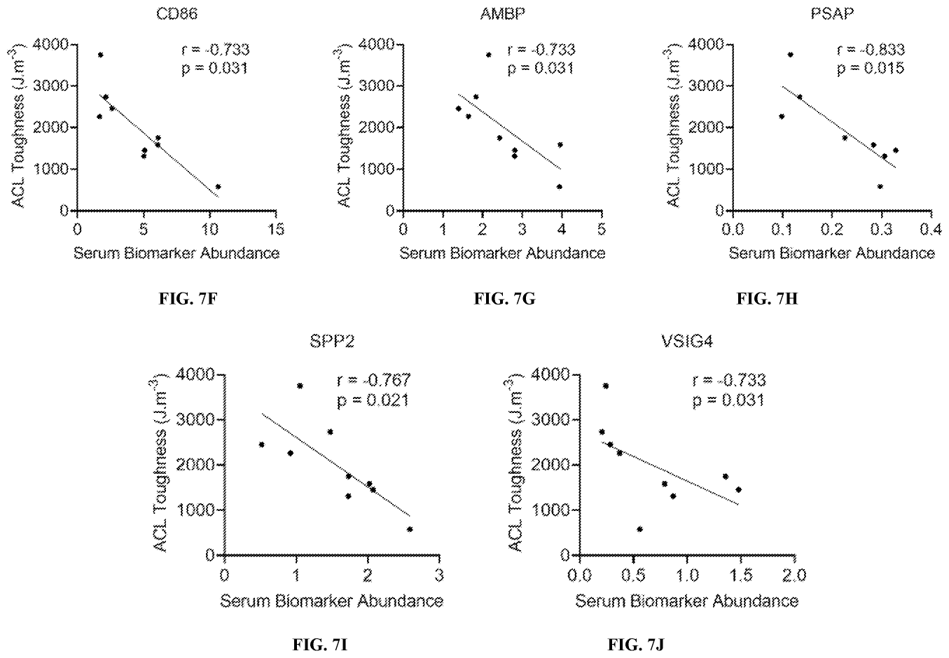

In MPS I, ACLs were found to exhibit functional abnormalities including reduced stiffness, toughness and failure properties compared to healthy controls (FIGS. 5A-5F). To determine whether candidate serum biomarkers could effectively predict diminished ACL biomechanical properties, Spearman correlation analyses were performed (Table 3). Most biomarkers exhibited strong, negative and significant (shown in bold) correlations with ACL biomechanical properties, including stiffness (FIGS. 6A-6J), toughness (FIGS. 7A-7J) and failure properties. These findings show that candidate serum biomarkers can effectively predict diminished ACL biomechanical properties and, by extension, abnormal knee joint function in MPS I dogs.

TABLE 2
List of proteins having abundance that was significantly different in both synovial fluid and in serum.

| | | log2FC | | log2FC | | | | | |
|---|---|---|---|---|---|---|---|---|---|
| | | Synoval | FC Synovial | Serum | FC Serum | | | | |
| | | Fluid MPS | Fluid MPS | MPS vs | MPS vs | | | | |
| Symbol | Full Name | vs Control | vs Control | Control | Control | Spearman r | P value | Pearson r | P value |
| AMBP | Alpha-1-microglobulin | 1.3647 | 2.5751 | 0.7780 | 1.7147 | 0.8727 | 0.0009 | 0.8163 | 0.0020 |
| AMY | Alpha-amylase | 1.2163 | 2.3235 | 1.0463 | 2.0652 | 0.7727 | 0.0074 | 0.8493 | 0.0009 |
| ANGPTL3 | Angiopoietin like 3 | 1.2123 | 2.3170 | 0.6024 | 1.5182 | 0.8455 | 0.0018 | 0.8200 | 0.0020 |
| AOC3 | Amine Oxidase Copper | −1.0179 | 0.4938 | −1.3586 | 0.3900 | 0.8182 | 0.0033 | 0.8712 | 0.0005 |
| | Containing 3 | | | | | | | | |
| APBB1 | Amyloid Beta Precursor | 1.1535 | 2.2246 | 0.7759 | 1.7122 | 0.8091 | 0.0039 | 0.8007 | 0.0031 |
| | Protein Binding Family B | | | | | | | | |
| | Member 1 | | | | | | | | |
| APOH | Beta-2-glycoprotein 1 | 0.6302 | 1.5478 | 0.3799 | 1.3012 | 0.7364 | 0.0128 | 0.7017 | 0.0161 |
| ATRN | Attractin | 0.7256 | 1.6536 | 0.6602 | 1.5803 | 0.7545 | 0.0098 | 0.7482 | 0.0080 |
| AZGP1 | Alpha-2-glycoprotein 1, | 1.1369 | 2.1991 | 0.9911 | 1.9877 | 0.8909 | 0.0005 | 0.8631 | 0.0006 |
| | zinc-binding | | | | | | | | |
| B2M | Beta-2-microglobulin | 0.9349 | 1.9118 | 1.0135 | 2.0188 | 0.7273 | 0.0144 | 0.6872 | 0.0195 |
| C2 | Complement C2 | 0.8606 | 1.8157 | 0.5260 | 1.4399 | 0.7818 | 0.0064 | 0.8147 | 0.0023 |
| C4A | C4a anaphylatoxin | 0.9664 | 1.9540 | 0.6416 | 1.5601 | 0.9000 | 0.0004 | 0.8571 | 0.0007 |
| C9 | Complement C9 | 0.7890 | 1.7278 | 0.8382 | 1.7878 | 0.6545 | 0.0336 | 0.7285 | 0.0110 |
| CA2 | Carbonic Anhydrase 2 | −2.2253 | 0.2139 | −0.7482 | 0.5953 | 0.6455 | 0.0368 | 0.5147 | 0.1052 |
| CANT1 | Calcium Activated | 0.8296 | 1.7772 | 1.5008 | 2.8300 | 0.8909 | 0.0005 | 0.8001 | 0.0031 |
| | Nucleotidase 1 | | | | | | | | |
| CD86 | CD86 molecule | 1.5613 | 2.9512 | 1.5937 | 3.0182 | 0.6364 | 0.0402 | 0.7221 | 0.0121 |
| CDH1 | Cadherin 1-like | 2.2159 | 4.6456 | 1.7458 | 3.3538 | 0.9455 | 0.0001 | 0.9606 | 0.0001 |
| CPN1 | Carboxypeptidase N | 1.2332 | 2.3510 | 0.9754 | 1.9662 | 0.8727 | 0.0009 | 0.8594 | 0.0007 |
| | subunit 1 | | | | | | | | |
| CPN2 | Carboxypeptidase N | 1.0431 | 2.0607 | 1.0004 | 2.0005 | 0.9545 | 0.0001 | 0.9080 | 0.0001 |
| | subunit 2 | | | | | | | | |
| CREB3L3 | cAMP responsive | 2.2936 | 4.9029 | 2.2610 | 4.7932 | 0.5476 | 0.1710 | 0.8723 | 0.0047 |
| | element binding protein 3 | | | | | | | | |
| | like 3 | | | | | | | | |
| CRP | C-reactive protein | 2.1170 | 4.3379 | 2.8869 | 7.3970 | 0.8818 | 0.0007 | 0.9035 | 0.0001 |
| EIF4A2 | Eukaryotic Translation | −1.1615 | 0.4470 | −1.0794 | 0.4732 | 0.7909 | 0.0055 | 0.7672 | 0.0059 |
| | Initiation Factor 4A2 | | | | | | | | |
| GANAB | Glucosidase II alpha | 0.7461 | 1.6772 | 1.9681 | 3.9126 | 0.5364 | 0.0939 | 0.6428 | 0.0329 |
| | subunit | | | | | | | | |
| ITIH2 | Inter-alpha-trypsin | 0.6167 | 1.5334 | 0.4622 | 1.3776 | 0.7091 | 0.0182 | 0.7487 | 0.0080 |
| | inhibitor heavy chain 2 | | | | | | | | |
| ITIH3 | Inter-alpha-trypsin | 1.9901 | 3.9725 | 1.9060 | 3.7476 | 0.9000 | 0.0004 | 0.9735 | 0.0001 |
| | inhibitor heavy chain 3 | | | | | | | | |
| LAMP2 | Lysosomal Associated | 2.3725 | 5.1783 | 0.9766 | 1.9679 | 0.6818 | 0.0251 | 0.6539 | 0.0291 |
| | Membrane Protein 2 | | | | | | | | |
| LBP | Lipopolysaccharide- | 1.2117 | 2.3162 | 1.1448 | 2.2111 | 0.9182 | 0.0020 | 0.9023 | 0.0001 |
| | binding protein | | | | | | | | |
| LRG1 | Leucine rich alpha-2- | 0.8016 | 1.7431 | 0.7110 | 1.6370 | 0.7455 | 0.0112 | 0.7408 | 0.0091 |
| | glycoprotein 1 | | | | | | | | |
| MAN2B1 | Alpha-mannosidase | 1.8351 | 3.5681 | 1.6660 | 3.1733 | 0.9000 | 0.0004 | 0.9382 | 0.0001 |
| MGAM | Maltase-glucoamylase | −0.8441 | 0.5571 | −1.0742 | 0.4749 | 0.9545 | 0.0001 | 0.8827 | 0.0003 |
| MMP19 | Matrix metalloproteinase | 3.3508 | 10.2022 | 1.2887 | 2.4431 | 0.7727 | 0.0074 | 0.8820 | 0.0003 |
| | 19 | | | | | | | | |
| ORM1 | Orosomucoid | 1.4664 | 2.7632 | 1.2113 | 2.3155 | 0.8000 | 0.0047 | 0.8843 | 0.0003 |
| PSAP | Prosaposin | 1.4455 | 2.7236 | 1.3543 | 2.5567 | 0.8424 | 0.0037 | 0.9402 | 0.0001 |
| PTPRG | Protein-tyrosine- | 0.8065 | 1.7490 | 0.7047 | 1.6298 | 0.8182 | 0.0033 | 0.7658 | 0.0060 |
| | phosphatase Receptor | | | | | | | | |
| | Type G | | | | | | | | |
| QSOX1 | Quiescin Sulfhydryl | 1.0906 | 2.1297 | 0.9577 | 1.9422 | 0.8545 | 0.0015 | 0.9011 | 0.0002 |
| | Oxidase 1 | | | | | | | | |
| RARRES2 | Chemerin | 1.4958 | 2.8202 | 1.2343 | 2.3526 | 0.8000 | 0.0047 | 0.8167 | 0.0022 |
| SERPINA3 | SERPIN domain- | 0.7135 | 1.6398 | 0.3389 | 1.2648 | 0.7273 | 0.0144 | 0.6135 | 0.0447 |
| | containing protein | | | | | | | | |
| SERPINA7 | Serpin family A member | 0.7135 | 1.6398 | 1.0375 | 2.0527 | 0.9273 | 0.0001 | 0.9447 | 0.0001 |
| | 7 | | | | | | | | |
| SPP2 | Secreted phosphoprotein | 1.6235 | 3.0812 | 1.2286 | 2.3434 | 0.8636 | 0.0012 | 0.8325 | 0.0015 |
| | 2 | | | | | | | | |
| TFRC | Transferrin receptor | −0.7436 | 0.5973 | −0.9118 | 0.5315 | 0.9182 | 0.0002 | 0.8437 | 0.0011 |
| | protein 1 | | | | | | | | |
| URAH | 5-hydroxyisourate | | | 1.48 | | | 0.0012 | | |
| | hydrolase | | | | | | | | |
| VSIG4 | V-set and | 1.7598 | 3.3865 | 1.7659 | 3.4009 | 0.6818 | 0.0251 | 0.6976 | 0.0170 |
| | immunoglobulin domain | | | | | | | | |
| | containing 4 | | | | | | | | |

TABLE 3
Correlations (Spearman) between candidate biomarkers of synovial joint disease measured in
serum and biomechanical properties of donor-matched anterior cruciate ligaments (ACLs).

| Serum | ACL Stiffness | | ACL Modulus | | ACL Failure Load | | ACL Failure Stress | | ACL Toughness | |
|---|---|---|---|---|---|---|---|---|---|---|
| Biomarker | r | p | r | p | r | p | r | p | r | p |
| MMP19 | −0.583 | 0.108 | −0.333 | 0.385 | −0.783 | 0.017 | −0.650 | 0.067 | −0.633 | 0.076 |
| ITIH3 | −0.900 | 0.002 | −0.733 | 0.031 | −0.850 | 0.006 | −0.900 | 0.002 | −0.933 | 0.001 |
| RARRES2 | −0.817 | 0.011 | −0.083 | 0.843 | −0.633 | 0.076 | −0.633 | 0.076 | −0.633 | 0.076 |
| CRP | −0.800 | 0.014 | −0.267 | 0.493 | −0.750 | 0.026 | −0.783 | 0.017 | −0.717 | 0.037 |
| MAN2B1 | −0.817 | 0.011 | −0.683 | 0.050 | −0.783 | 0.017 | −0.783 | 0.017 | −0.833 | 0.008 |
| CD86 | −0.783 | 0.017 | −0.500 | 0.178 | −0.850 | 0.006 | −0.800 | 0.014 | −0.733 | 0.031 |
| AMBP | −0.917 | 0.001 | −0.583 | 0.108 | −0.683 | 0.050 | −0.700 | 0.043 | −0.733 | 0.031 |
| PSAP | −0.643 | 0.096 | −0.452 | 0.268 | −0.881 | 0.007 | −0.643 | 0.096 | −0.833 | 0.015 |
| SPP2 | −0.917 | 0.001 | −0.550 | 0.133 | −0.817 | 0.011 | −0.800 | 0.014 | −0.767 | 0.021 |
| VSIG4 | −0.683 | 0.050 | −0.433 | 0.250 | −0.733 | 0.031 | −0.800 | 0.014 | −0.733 | 0.031 |

### Example 4. Exhibit A—Proteomics Identifies Novel Biomarkers of Synovial Joint Disease in a Canine Model of Mucopolysaccharidosis I—Summary of Examples 1-3

### Abstract

Mucopolysaccharidosis I is a lysosomal storage disorder characterized by deficient alpha-L-iduronidase activity, leading to abnormal accumulation of glycosaminoglycans in cells and tissues. Synovial joint disease is prevalent and significantly reduces patient quality of life. There is a critical need for improved understanding of joint disease pathophysiology in MPS I, including specific biomarkers to predict and monitor joint disease progression, and response to treatment. The objective of this study was to leverage the naturally-occurring MPS I canine model and undertake an unbiased proteomic screen to identify systemic biomarkers predictive of local joint disease in MPS I. Synovial fluid and serum samples were collected from MPS I and healthy dogs at 12 months-of-age, and protein abundance characterized using liquid chromatography tandem mass spectrometry. Stifle joints were evaluated postmortem using magnetic resonance imaging (MRI) and histology. Proteomics identified 40 proteins for which abundance was significantly correlated between serum and synovial fluid, including markers of inflammatory joint disease and lysosomal dysfunction. Elevated expression of three biomarker candidates, matrix metalloproteinase 19, inter-alpha-trypsin inhibitor heavy-chain 3 and alpha-1-microglobulin, was confirmed in MPS I cartilage, and serum abundance of these molecules was found to correlate with MRI and histological degenerative grades. The candidate biomarkers identified have the potential to improve patient care by facilitating minimally-invasive, specific assessment of joint disease progression and response to therapeutic intervention.

### Introduction

The mucopolysaccharidoses (MPS) are a family of inherited lysosomal storage disorders characterized by deficiencies in enzymes that degrade glycosaminoglycans (GAGs) [1]. These GAGs accumulate in cells and tissues resulting in progressive, multi-organ disease manifestations. There are 11 distinct types of MPS, each characterized by a unique enzyme deficiency resulting from a mutation in the associated gene [1]. The types of GAGs that accumulate also vary between MPS types, resulting in a spectrum of clinical manifestations. The incidence in the United States across all subtypes is estimated to be approximately 1 in 100,000 births [2]. While multiple organ systems are affected, skeletal manifestations are present in all MPS subtypes with varying degrees of severity [3]. Referred to collectively as dysostosis multiplex, these manifestations may include spinal deformity, short stature and synovial joint abnormalities [4-7]. Skeletal manifestations range from very mild to severe and in some cases require surgical correction [8, 9].

MPS I, also called Hurler Syndrome, or Hurler-Scheie or Scheie in its attenuated forms, is characterized by deficient α-L-iduronidase (IDUA) activity, leading to progressive accumulation of poorly degraded heparan and dermatan sulfate GAGs in multiple cell and tissue types [10]. Joint abnormalities in MPS I patients are prevalent, and patients experience significantly decreased quality of life due to pain and mobility impairment. Reported characteristics of joint disease include stiffness and limited range of motion, thought to result from abnormalities in the ligaments, joint capsules, and underlying epiphyseal bone [11, 12]. Almost all joints are involved, with the earliest presentation in shoulders, hands and knees [11, 12]. Patients report pain and difficulty completing daily activities, and symptoms in undiagnosed patients may be mistaken for inflammatory rheumatic disorders [12]. Relatively little is known about the molecular pathophysiology of joint disease in MPS. Common characteristics across several disease subtypes, including MPS I, VI and VII, include inflammation-mediated articular cartilage destruction, impaired epiphyseal bone formation, and arthritic-like joint changes, all of which occur downstream of GAG accumulation [13-15].

Current clinical treatments for MPS I patients include enzyme replacement therapy (ERT) and hematopoietic stem cell transplantation; however, while these treatments effectively increase patient lifespan, they often fail to prevent progression of skeletal disease [16, 17]. Emerging therapeutic strategies such as gene therapy hold significant promise; however, sensitive and specific biomarkers for assessing the response of joint disease in clinical trials are lacking. Biomarkers are also essential for effective early diagnosis and monitoring of joint disease progression in MPS I patients. Application of gold standard radiological assessments, such as magnetic resonance imaging (MRI), is often challenging in MPS I patients due to their young age and cognitive impairment, and may necessitate potentially risky general anesthesia [18]. Protein biomarkers that can be measured systemically, for example in serum, plasma, urine or saliva, are a promising alternative. A handful of studies have used unbiased screening techniques to identify biomarkers of MPS more broadly, focusing on a limited number of subtypes [19-22]. There have also been more targeted studies that have sought to leverage the reported role of inflammation in MPS joint disease [13, 23]. For example, a recent study established correlations between inflammatory molecules present in plasma and urine, and degree of joint dysfunction in MPS patients [23]. To our knowledge, there have been no studies that have employed unbiased screening to identify molecular biomarkers specific for synovial joint disease in any MPS subtype. The naturally-occurring canine model of MPS I mimics the progressive synovial joint changes and decline in mobility that occur in human patients, and represents a powerful, clinically-relevant model for biomarker discovery and validation. The objective of this study was to leverage this model and undertake an unbiased proteomic screen to identify systemic molecular biomarker candidates predictive of local synovial joint disease in MPS I dogs.

### Materials and Methods

### Animals and Sample Collection

All animal work was conducted with approval from the University of Pennsylvania Institutional Animal Care and Use Committee. For this study, we used the naturally-occurring MPS I canine model. There were two study groups: MPS I-affected dogs (n=6; 3 males and 3 females) and healthy controls (n=5; 2 males and 3 females). MPS I dogs have a homozygous donor splice site mutation in intron 1 of the IDUA gene [24] and are considered to align most closely with the intermediate severity Hurler-Scheie phenotype found in human patients, based primarily on observed pathological manifestations in the central nervous system, skeleton and corneas [25, 26]. MPS I-affected animals were identified at birth by DNA mutation analysis. Control animals were heterozygous (phenotypically normal) littermates of MPS I dogs. Animals were raised and housed at the University of Pennsylvania School of Veterinary Medicine under NIH and USDA guidelines for the care and use of animals in research. Animals were housed in kennel runs in groups of 2 or 3, with a light cycle of 12 hours per day and an ambient temperature of 21° C., with food and water provided ad libitum.

Physical examinations were performed monthly by a veterinarian (MLC), and included general clinical evaluations as well as specific assessments of mobility, gait, joint swelling and muscle tone. At 12 months-of-age, all animals were euthanized via an overdose of sodium pentobarbital consistent with the recommendations of the American Veterinary Medical Association. This terminal timepoint was selected as MPS I dogs typically manifest clear ambulatory deficits between 8 and 12 months-of-age. Prior to euthanasia, blood was collected from the cephalic vein, let sit for 30 min at room temperature until blood clots formed, then spun for 10 min in a refrigerated centrifuged at 3000 RCF for serum separation. Immediately following euthanasia, the right stifle (knee) joint was opened and synovial fluid (approximately 200 μl) was aspirated using an 18-gauge needle. Both serum and synovial fluid samples were then aliquoted, snap frozen in liquid nitrogen, and stored at −80° C. The left stifle joint was collected, sealed in plastic and stored at −20° C. for subsequent MRI and histological evaluation.

### Proteomics

The detailed proteomics methodology is provided as supplementary information. Serum and synovial fluid samples were prepared for liquid chromatography tandem mass spectrometry (LC/MS/MS) analysis by solubilization, reduction, and alkylation followed by digestion with trypsin. The desalted samples were randomized and injected onto a Thermo QEHF mass spectrometer and collected using data independent acquisition. Spectronaut (version 15) [27] was employed for protein identification and quantification, using the reference Canus Lupus Familiaris proteome from Uniprot. Perseus (1.6.14.0) [28] was employed for data processing and statistical analysis using the MS2 intensity values generated by Spectronaut. The data were log 2 transformed and normalized by subtracting the median for each sample. Statistical analysis of acquired data was performed as described below. The proteins that were exclusively detected in one experimental group were also reported for further bioinformatics analysis. Gene ontology (GO) and Kyoto Encyclopedia of Genes and Genomes (KEGG) pathway enrichment analyses were performed for both synovial fluid and serum results.

### Magnetic Resonance Imaging

Left stifle joints were thawed at 4° C. overnight and brought to room temperature, and imaged with musculature and skin intact using a 3T MR scanner (Magnetom Trio with TIM system; Siemens Healthcare, Malvern, PA, USA) and a 4-channel small flex coil, with the joint at an anatomical angle of 135° [29]. Three sequences were acquired: T2-weighted with fat suppression (TR/TE=8050/77 ms); proton density-weighted (PD; TR/TE=3900/40 ms); and T1-weighted volumetric interpolated breath-hold examination with water excitation (T1-VIBE; TR/TE=10.4/4.9 ms). The slice thickness was 1.5 mm and field of view 140 mm for all sequences, and images were acquired in both the sagittal and dorsal (coronal) planes. For semi-quantitative assessment of joint pathology, a grading scheme was adapted using elements of both the Knee Osteoarthritis Scoring System (KOSS) and MRI Osteoarthritis Knee Score (MOAKS) schemes [30, 31], inclusive of the following features: effusion synovitis (fluid effusions anterior/cranial to the joint), Baker's cysts (fluid effusions posterior/caudal to the joint), meniscal degeneration, meniscal extrusion, patellar displacement, fat pad synovitis, bone marrow edema, cartilage defects and subchondral cysts. Each feature was graded from 0 (absent) to 3 (severe) by three independent, blinded assessors. Overall grade was calculated as the sum of individual grades. Scores from individual reviewers were averaged prior to statistics.

### Immunohistochemical Validation of Local Expression in Articular Cartilage

Following MRI, the distal femur was isolated from the left stifle joint, fixed in 10% neutral buffered formalin for 1 week and completely decalcified in formic acid/ethylenediaminetetraacetic acid (Formical 2000; Statlab, Louisville, USA). A 3 mm-thick mid-sagittal slice was then cut from the medial femoral condyle and processed into paraffin. Protein expression levels of three candidate biomarkers identified from the proteomic screen (matrix metalloproteinase 19 (MMP19), alpha-1-microglobulin (A1M) and inter-alpha-trypsin inhibitor heavy chain 3 (ITIH3)) in cartilage were examined using immunohistochemistry. Sections of healthy canine lung were used as a positive control for MMP19, while liver was used as a positive control for both A1M and ITIH3. Primary antibodies were purchased from Abcam (Cambridge, United Kingdom; catalog numbers ab53146, ab47980 and ab 197188 for MMP19, A1M and ITIH3, respectively). Antigen retrieval was carried out on rehydrated sections using a heat mediated technique in a bath of Tris-EDTA pH 8.0 buffer for 15 minutes at 95° C. Sections were treated with 3% hydrogen peroxide followed by Background Buster (Innovex Biosciences; Richmond, USA), and then incubated with each primary antibody (1:200 for all) overnight at 4° C. Antibody staining was visualized using the Vectastain Elite ABC-Peroxidase Kit (Vector Laboratories; Burlingame, USA) and diaminobenzidine chromogen (Thermo Fisher Scientific; Waltham, USA). Finally, sections were counter stained with hematoxylin QS (Vector Laboratories) and cover-slipped with aqueous mounting medium (Agilent; Santa Clara, USA). For analysis, all slides were imaged under bright field light microscopy (Eclipse 90i; Nikon; Tokyo, Japan). For quantitative assessment of cell immunopositivity, three randomly selected regions of interest per section at 10× magnification and spanning the full cartilage thickness were analyzed. For each region, the number of immunopositive cells was counted and normalized as a percentage of the total number of cells present. Only cells with a nucleus were counted (i.e. empty lacunae were excluded). Cell counting for each region was performed by three individuals who were blinded to the study groups, averaged and taken as a single biological replicate for statistical comparisons.

### Histological Grading of Articular Cartilage Condition

Histological sections 7 μm thick were double-stained with safranin O and fast green, and imaged under bright field light microscopy. Semi-quantitative grading of cartilage condition was performed using an adaptation of the OARSI guidelines for canine osteoarthritis by three independent and blinded assessors [32]. The following parameters were determined: cartilage structure (surface undulations/fissures), proteoglycan loss, and chondrocyte pathology (cell enlargement, increased cell numbers and/or increased cell clustering). Each section was divided into thirds across the width of the condyle, and graded for local (1/3) multifocal (2/3) or global (3/3) pathology in each category, with scores ranging from 0 for completely healthy to 12 for severely degenerate [32]. Overall grade (sum of individual parameters) was also calculated. The scores from individual reviewers were averaged prior to statistics.

### Statistical Analyses

For proteomics data, Perseus software was used to establish statistically-significant fold changes in protein abundance in MPS I vs control samples. Student's t-tests were employed to identify differentially abundant proteins using adjusted p values<0.05 as the significance threshold. For proteins that were significantly different (MPS I vs control) for both serum and synovial fluid, correlations between the abundance (normalized log 2 intensity) of each protein in serum and the same protein in synovial fluid were assessed using Pearson's tests. Normality of data distribution was confirmed using Shapiro-Wilk tests. Where data were normally distributed, significant differences in cartilage cell immunopositivity, histological grading parameters, and joint MRI grading parameters between control and MPS I were established using unpaired t-tests, and data presented as mean±standard deviation. Where data were not normally distributed, Mann-Whitney tests were used, and data presented as median and interquartile range. Correlations between abundance (normalized log 2 intensity) of selected biomarker candidates in both synovial fluid and serum, and overall cartilage and MRI grades were determined using Spearman's rank order tests. P<0.05 was considered statistically significant for all tests.

### Results

### Clinical Findings

All animals reached the study end point of 12 months-of-age without any adverse events. Animals received comprehensive weekly physical examinations, which included clinical assessments of joint function and ambulatory ability. Throughout the duration of the study, no abnormalities were noted in control dogs. General clinical signs of disease in MPS I animals included corneal clouding that was mild progressing to moderate throughout the study, prominent third eyelids, erythematous conjunctivae, prognathism inferior, and thickened skin under the chin. All MPS I animals had reducible umbilical hernias, and 5 of 6 animals developed systolic heart murmurs between 5 and 8 months of age ranging in severity from I/VI to II/VI. With respect to skeletal disease manifestations, all MPS I animals were ambulatory throughout the study period; however, all had hyperextended carpi, stifles, and tarsi and widely splayed digits. Five of the 6 MPS I animals developed mild to moderate joint effusion between 2 and 7 months of age. The joints were neither painful nor warm to the touch.

### Proteomics

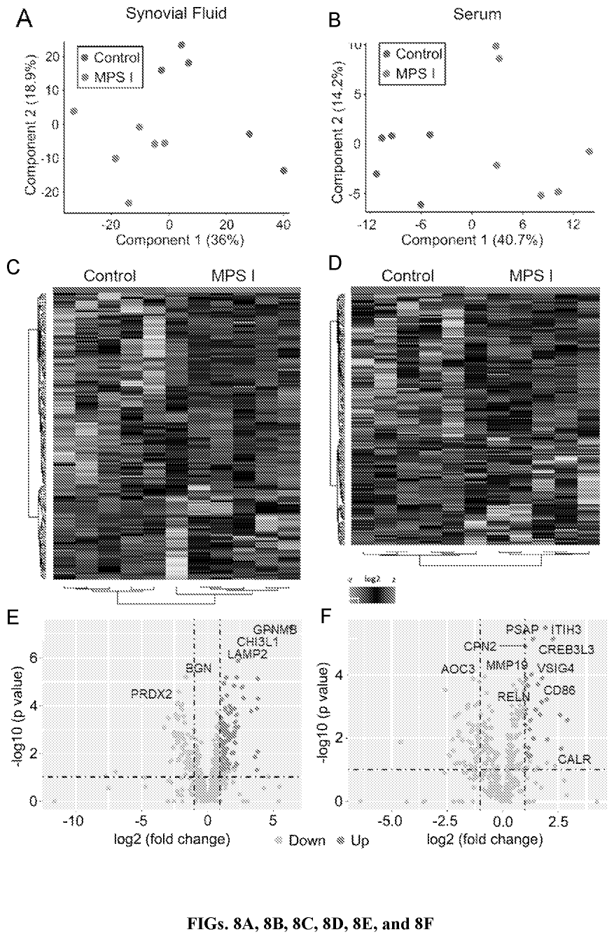

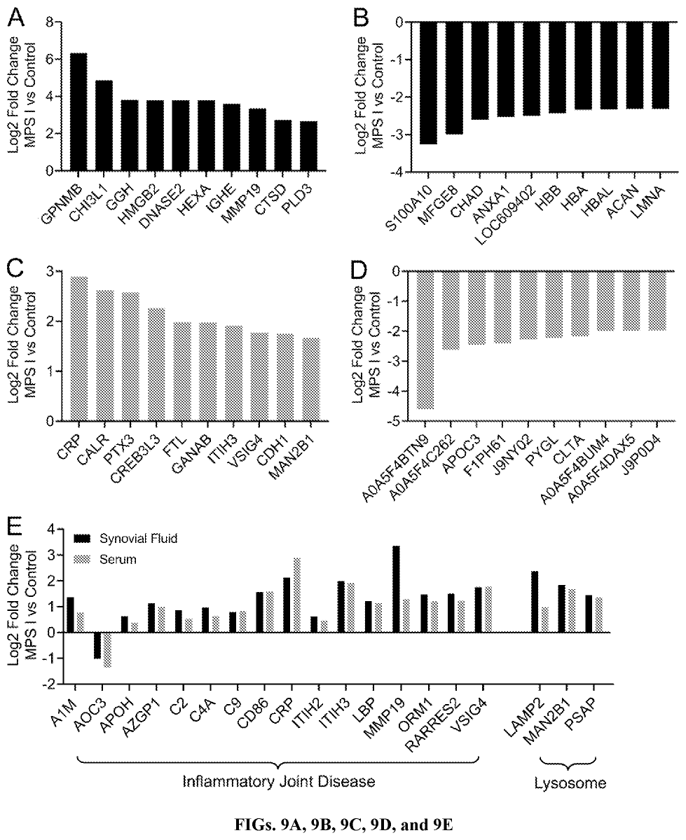

For both synovial fluid and serum, principal component analyses (FIGS. 8A and 8B) demonstrated clustering of control and MPS I samples, confirming significant effects of disease state on relative protein abundance in both sample types. In synovial fluid, a total of 810 unique proteins were identified, of which 149 exhibited significantly different abundance in MPS I versus control (FIGS. 8C-8F). Of these proteins, 101 were significantly upregulated (Table 5), and 48 were significantly downregulated (Table 6). In serum, a total of 410 unique proteins were identified, of which 150 exhibited significantly different abundance in MPS I vs control (FIGS. 8C-8F). Of these proteins, 85 were significantly upregulated (Table 7), and 65 were significantly downregulated (Table 8). The top 10 upregulated and down regulated proteins in both sample types are shown in FIGS. 9A-9D. In synovial fluid, the top upregulated protein was transmembrane glycoprotein NMB (GPNMB, log 2 fold change 6.32 in MPS I versus control, p<0.001). In serum the top upregulated protein was c-reactive protein (CRP, log 2 fold change 2.89 in MPS I versus control, p=0.003). To identify candidate circulating biomarkers predictive of local synovial joint disease, we examined correlations between molecules with significantly differential abundance in both synovial fluid and serum. There were 50 proteins for which abundance was significantly different for both SF and serum, and of those, 40 exhibited a significant correlation in abundance (normalized log 2 intensity) between synovial fluid and serum across all 11 samples (Table 4). Of these 40 candidate biomarkers, 19 were identified as being associated with inflammatory joint disease or lysosomal dysfunction based on an examination of prior literature (FIG. 9E).

### Pathway Analysis

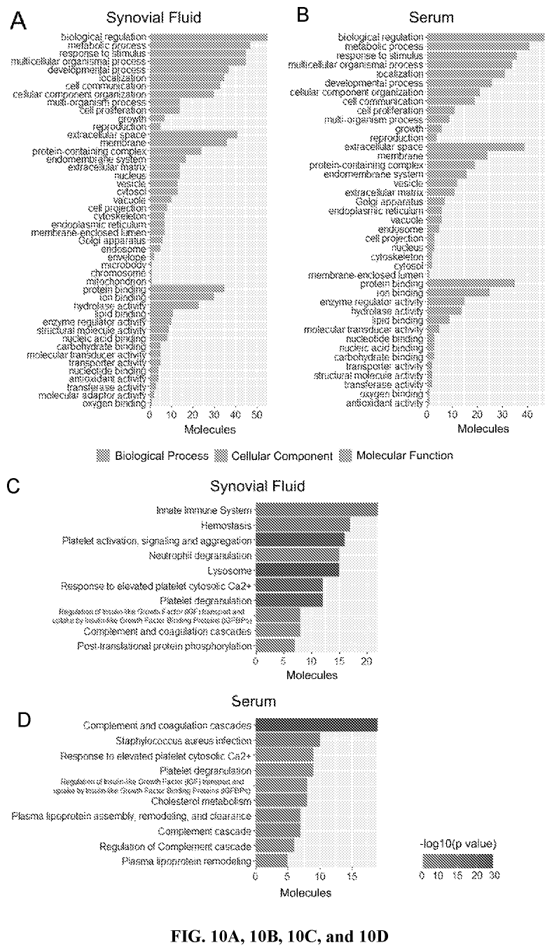

For both synovial fluid and serum, the top GO terms (by number of molecules) were biological regulation, extracellular space and protein binding, for biological process, cellular component and molecular function, respectively (FIGS. 10A and 10B). Metabolic process was the second top biological process term for both sample types. With respect to KEGG analysis, the top enriched pathway for synovial fluid was innate immune system (FIGS. 10C and 10D). Other notable pathways amongst the top 10 included lysosome, and complement and coagulation cascade. For serum, the top enriched pathway was complement and coagulation cascade, while complement cascade and regulation of complement cascade were also among the top 10 pathways. The regulation of insulin-like growth factor (IGF) transport and uptake by insulin-like growth factor binding proteins (IGFBPs) pathway was also enriched for both sample types.

### Immunohistochemical Validation of Local Expression in Articular Cartilage

Three candidate biomarkers identified through proteomic screening were selected for validation of local expression in stifle joint articular cartilage: matrix metalloproteinase 19 (MMP19), inter-alpha-trypsin inhibitor heavy chain 3 (ITIH3) and alpha-1-microglobulin (A1M). All three of these molecules exhibited significant upregulation in both synovial fluid and serum (FIG. 9E), and significant correlation in abundance between synovial fluid and serum (Table 4). For ITIH3, the percentage of immunopositive cells was 2.5-fold higher in MPS I cartilage compared to control (p=0.004, FIGS. 11A and 11B). For MMP19, percentage of immunopositive cells was 2.6-fold higher in MPS I cartilage compared to control (p=0.009, FIGS. 11C and 11D). Finally, for A1M, the percentage of immunopositive cells was 2.0-fold higher in MPS I cartilage compared to control (p=0.003, FIGS. 11E and 11F).

### Magnetic Resonance Imaging

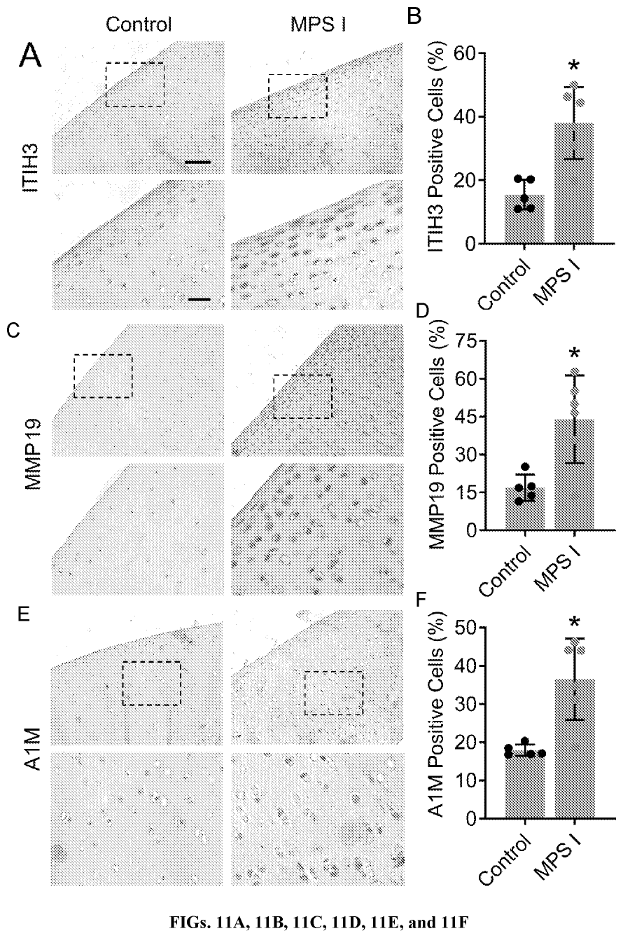

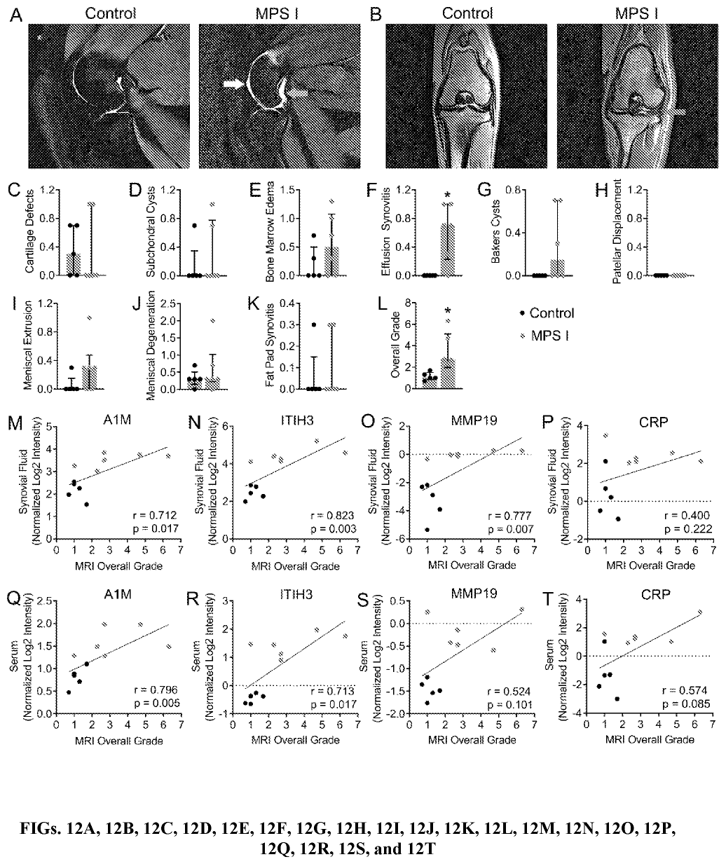

Representative stifle joint MRI images (sagittal and dorsal) for control and MPS I animals are shown in FIGS. 12A and 12B. Elevated fluid cranial and caudal to the joint was frequently observed in MPS I animals compared to controls. Meniscal intrasubstance degeneration and extrusion were occasionally observed, while articular cartilage structure appeared normal for most animals. Semi-quantitative grading (FIGS. 12C-12L) revealed significantly worse effusion synovitis and overall MRI grade for MPS I animals compared to controls (p=0.015 and 0.024, respectively), while other individual grading parameters were not significantly different. In synovial fluid, A1M, ITIH3 and MMP19 abundance were all significantly and positively correlated with overall MRI grade (r=0.712, p=0.017; r=0.823, p=0.003; and r=0.777, p=0.007, respectively, FIGS. 12M-12O), while CRP (the top upregulated molecule in serum) was not (r=0.400, p=0.222; FIG. 12P). In serum, A1M and ITIH3 abundance were significantly and positively correlated with overall MRI grade (r=0.796, p=0.005; and r=0.713, p=0.017, respectively, FIGS. 12Q and 12R), while MMP19 and CRP were not (r=0.524, p=0.101; and r=0.574, p=0.085, respectively; FIGS. 12S and 12T).

### Histological Grading of Articular Cartilage Condition

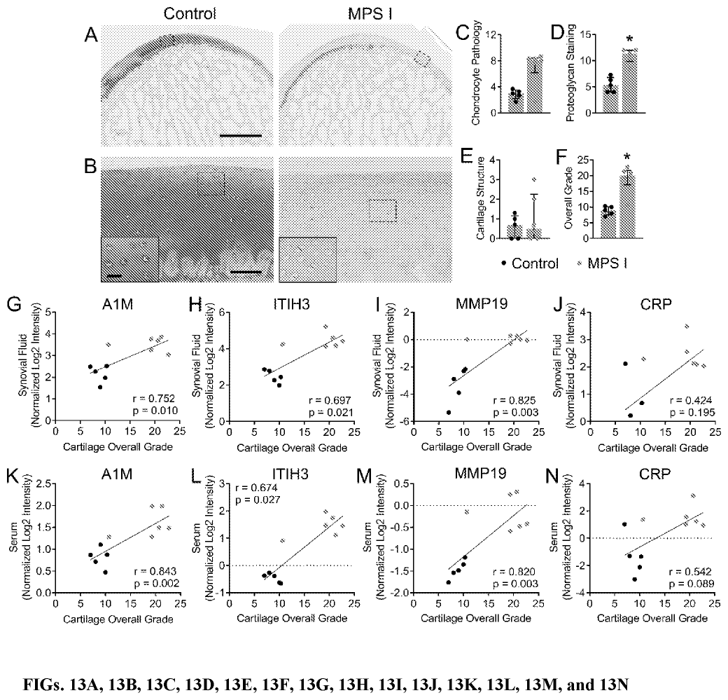

Representative images of safranin-O and fast green double-stained sections of articular cartilage from the medial femoral cartilage of control and MPS I animals are shown in FIGS. 13A and 13B. Qualitatively, MPS I cartilage appeared moderately degenerated, including generalized loss of proteoglycan staining, and cell clustering and enlargement, while the surface structure was generally intact. Semi-quantitative grading revealed that cartilage from MPS I animals exhibited significantly worse proteoglycan staining and overall grade (p=0.004 for both), compared to control cartilage, while chondrocyte pathology and cartilage structure were not significantly different (p=0.076 and p=0.992, respectively, FIGS. 13C-13F). In synovial fluid, A1M, ITIH3 and MMP19 abundance were all significantly and positively correlated with overall cartilage grade (r=0.752, p=0.010; r=0.697, p=0.021; and r=0.825, p=0.003, respectively, FIGS. 13G-13I), while CRP was not (r=0.424, p=0.195; FIG. 13J). Similarly, in serum, A1M, ITIH3 and MMP19 abundance were significantly and positively correlated with overall cartilage grade (r=0.843, p=0.002; r=0.674, p=0.027; and r=0.820, p=0.003, respectively, FIGS. 13K-13M), while CRP was not (r=0.542, p=0.089; FIG. 13N).

### Discussion

Many patients with MPS exhibit progressive and debilitating synovial joint disease that impairs their mobility and capacity for independent living. For reasons still not well understood, the severity of joint disease varies markedly both between and within MPS subtypes. Molecular biomarkers that detect early pathological changes in the joints of MPS patients would represent a powerful tool to be used in their clinical management, including assessing early response to therapeutic intervention. As MPS patients may be initially misdiagnosed as having other rheumatic disorders, such biomarkers may also serve as a first-line diagnostic assay. In this study, we leveraged a clinically relevant canine model in combination with unbiased proteomic screening to identify molecules that may serve as circulating biomarkers for joint disease in MPS I. By screening donor-matched synovial fluid and serum samples, we were able to identify a shortlist of 40 biomarker candidate molecules that exhibited high correlation between local and systemic abundance. We confirmed tissue-level expression of three biomarker candidates, the abundance of which correlated positively with joint degenerative condition.

MRI and histological findings in this study implicate multiple component joint tissues in the etiology of joint disease in MPS I dogs, and the associated pathological changes share a number of similarities with more common arthritic diseases. For example, fluid effusions are commonly observed in the knees of osteoarthritis patients, are associated with inflammation of the synovium (synovitis), and often correlate with painful symptoms [33]. In articular cartilage, observed altered cellularity and diminished proteoglycan staining are also hallmarks of osteoarthritis, where they are associated with local inflammatory changes [34]. Similar findings were recently reported for MPS VII dog articular cartilage at 6 months-of-age [35]. Inflammation has been previously implicated in the etiology of MPS joint disease across several subtypes, including MPS I, in both human patients and animal models, and is due in part to activation of the innate immune system by accumulating GAG fragments that function as damage-associated molecular pathogens [13, 14, 23, 35-38]. Indeed, in the current study, pathway analysis identified the innate immune system as the top enriched pathway for MPS I synovial fluid. Previous studies in MPS I mice identified cartilage proteoglycan loss, elevated matrix metalloproteinase expression and abnormal morphology as characteristics of progressive joint disease [37, 39]. Clinically, elevated tumor necrosis factor alpha (TNF-α) was found to be associated with increased pain and disability in MPS patients [38]. Interleukin 1-beta (IL-1β) and TNF-α were found to be significantly elevated in MPS I patient plasma but not correlate with joint dysfunction, while interleukin-6 (IL-6), was found to exhibit a significant association with decreased joint range of motion over time [23].

In the current study, we identified a number of molecules upregulated in both MPS I synovial fluid and serum that have previously been explored as circulating biomarkers for more common arthritic diseases. The top upregulated molecule found in MPS I serum, c-reactive protein (CRP), is an acute phase protein secreted by hepatocytes, immune cells and other cell types that is used as a systemic marker of inflammation in rheumatoid arthritis patients, where serum levels have been shown to correlate with local, tissue-level inflammatory changes [40]. CRP as a biomarker of osteoarthritis is less-well established, but has been shown to be elevated and associated with pain and loss of physical function [41]. Chemerin (RARRES2) is a chemoattractant adipokine implicated in immune and metabolic homeostasis [42]. Serum chemerin levels are elevated in rheumatoid arthritis patients and correlate with joint swelling [43]. Chemerin is also elevated in the synovial fluid, synovium and articular cartilage of knee osteoarthritis patients, and correlates with disease severity [44-46]. Taken together, our findings suggest that MPS I joint disease pathophysiology shares common elements with both osteoarthritis and rheumatoid arthritis, and that repurposing drugs currently prescribed or under development for these diseases may be a promising strategy for treating MPS I joint disease. Notably, some inflammatory molecules identified in prior studies such as the cytokines IL-6, IL-1β and TNF-α were not found in our results. This likely reflects a limitation of our proteomics screen, which due to the high dynamic range, meant that molecules present in relatively low abundance may not have been detected, even though they may nonetheless be important disease mediators.

We also confirmed tissue-level expression in MPS I articular cartilage for three molecules, MMP19, ITIH3 and A1M, with less-well established roles in arthritic diseases. Serum and synovial fluid abundance of these molecules was found to significantly and positively correlate with overall joint and/or cartilage degenerative condition assessed using MRI and histology, respectively. MMP19 exhibited elevated local expression in MPS I cartilage, and while this enzyme has not been extensively studied in the context of joint disease, it is able to autoactivate and degrade important extracellular matrix components such as aggrecan and cartilage oligomeric matrix protein [47, 48]. MMP19 was previously found to be elevated in sera from patients with rheumatoid arthritis and systemic lupus erythematosus [49]. The inter-alpha-trypsin inhibitors (ITIs) have important roles in regulating inflammation and extracellular matrix stabilization, including binding hyaluronic acid [50]. ITI proteins are comprised of a light chain (bikunin, a chondroitin sulfate proteoglycan) and multiple heavy chains, such as ITIH3 [51]. There is evidence that the concentration of hyaluronic acid-bound ITIs is elevated in the synovial fluid from rheumatoid arthritis patients [52]. Interestingly, we found that ITIH3 expression was particularly pronounced in the superficial zone of MPS I cartilage, and it has been suggested that ITIs may have a role in the protection of the articular surface [53]. AIM is a member of the lipocalin protein family, and has a central role in the cleaning of oxidative waste products, macromolecular repair, and antioxidation protection [54]. Increased levels of A1M are present in the synovial fluid and serum of acute inflammatory arthritis patients [55], and there is evidence that increased oxidative stress in MPS contributes to disease progression in multiple tissues, including cartilage [56, 57].

The top upregulated molecule in synovial fluid was GPNMB (also called osteoactivin), a trans-membrane glycoprotein expressed by numerous cell types including chondrocytes [58, 59]. GPNMB is positive regulator of osteoblast differentiation [60] and also been linked to lysosomal dysfunction [36]. Elevated GPNMB expression has been linked to several lysosomal storage disorders, where it has been proposed as a potential biomarker [61-65]. Previously, we demonstrated elevated mRNA expression of GPNMB in the vertebral epiphyseal cartilage of very young MPS VII dogs [66]. In the current study, while GPNMB was upregulated in synovial fluid, similar upregulation was not found serum, suggesting it may not be a suitable systemic biomarker candidate for joint disease.

For biomarkers to serve as a first-line diagnostic tool in previously undiagnosed patients, they would need to be able to effectively distinguish between joint disease associated with MPS I versus other rheumatic disorders. Notably, several lysosome-related molecules including lysosome-associated membrane protein 2 (LAMP2), alpha-mannosidase (MAN2B1) and prosaposin (PSAP) were found to be upregulated in both MPS I synovial fluid and serum, and exhibited significant correlation between local and systemic expression. A panel of serum biomarkers that includes both markers of inflammatory joint disease and markers of lysosomal dysfunction could potentially represent a molecular signature unique to joint disease in MPS I.

In addition to the innate immune system, pathway analysis identified complement cascade-related pathways as enriched in MPS I in both synovial fluid and serum. The complement cascade is a first line of defense in innate immunity, and consists of more than 50 circulating and cell-associated proteins [67]. The complement system is important in the pathophysiology of osteoarthritis, and complement components are elevated in the synovial fluid of patients in the early stages of the disease [67]. Local activation of complement in osteoarthritic joints is thought to occur in part due to the release of extracellular matrix degradation components and cellular debris from cartilage and synovial membrane [67]. Complement activation is also implicated in the pathogenesis of joint disease in rheumatoid arthritis patients [68]. Previously, in MPS VII mice, complement activation was shown to contribute to the pathogenesis of aortic dilatation [69]. Here, we provide evidence that complement activation may contribute to the progression of synovial joint disease in MPS I. Drugs that inhibit complement components are approved for treating several autoimmune disorder [70], and may represent a possible strategy for treating inflammatory disease in MPS I.

Limitations of this study include the afore-mentioned high dynamic range of the proteomic screen that may have precluded detection of some low abundance molecules. Tissue level validation was only performed for a small number of molecules and only in articular cartilage. A relatively small cohort of animals was studied, precluding an analysis of sex-dependent effects, and the capacity of biomarkers to predict joint disease severity within the MPS I cohort alone. Future studies will expand validation studies to additional molecules and joint tissues, and establish the potential for biomarkers to predict functional as well as structural changes. In conclusion, in this study we leveraged the clinically relevant canine model and unbiased proteomic screening to identify a panel of novel candidate biomarkers for synovial joint disease in MPS I. These biomarkers have the potential to improve patient care by facilitating minimally-invasive, specific assessment of joint disease progression and response to therapeutic intervention.

### References for Example 4

[1] E. F. Neufeld, J. Muenzer, The Mucopolysaccharidoses, in: C. R. Scriver, A. L. Beaudet, W. S. Sly, D. Valle (Eds.), The metabolic and molecular bases of inherited disease, McGraw-Hill, New York, 2001, pp. 3421-3452.
[2] Y. Puckett, A. Mallorga-Hernandez, A. M. Montano, Epidemiology of mucopolysaccharidoses (MPS) in United States: challenges and opportunities Orphanet J Rare Dis 16 (2021) 241.
[3] K. K. White, Orthopaedic aspects of mucopolysaccharidoses Rheumatology 50 (2011) V26-V33.
[4] S. Palmucci, G. Attina, M. L. Lanza, G. Belfiore, G. Cappello, P. V. Foti, P. Milone, D. Di Bella, R. Barone, A. Fiumara, G. Sorge, G. C. Ettorre, Imaging findings of mucopolysaccharidoses: a pictorial review Insights Imaging 4 (2013) 443-459.
[5] J. Muenzer, The mucopolysaccharidoses: a heterogeneous group of disorders with variable pediatric presentations J Pediatr 144 (2004) S27-34.
[6] K. Morishita, R. E. Petty, Musculoskeletal manifestations of mucopolysaccharidoses Rheumatology (Oxford) 50 Suppl 5 (2011) v19-25.
[7] A. M. Montano, N. Lock-Hock, R. D. Steiner, B. H. Graham, M. Szlago, R. Greenstein, M. Pineda, A. Gonzalez-Meneses, M. Coker, D. Bartholomew, M. S. Sands, R. Wang, R. Giugliani, A. Macaya, G. Pastores, A. K. Ketko, F. Ezgu, A. Tanaka, L. Arash, M. Beck, R. E. Falk, K. Bhattacharya, J. Franco, K. K. White, G. A. Mitchell, L. Cimbalistiene, M. Holtz, W. S. Sly, Clinical course of sly syndrome (mucopolysaccharidosis type VII) J Med Genet 53 (2016) 403-418.
[8] M. H. van der Linden, M. C. Kruyt, R. J. Sakkers, T. J. de Koning, F. C. Oner, R. M. Castelein, Orthopaedic management of Hurler's disease after hematopoietic stem cell transplantation: a systematic review J Inherit Metab Dis 34 (2011) 657-669.
[9] E. Ashby, D. Eastwood, Characterization of knee alignment in children with mucopolysaccharidosis types I and II and outcome of treatment with guided growth J Child Orthop 9 (2015) 227-233.
[10] C. S. Hampe, J. B. Eisengart, T. C. Lund, P. J. Orchard, M. Swietlicka, J. Wesley, R. S. McIvor, Mucopolysaccharidosis Type I: A Review of the Natural History and Molecular Pathology Cells 9 (2020).
[11] N. Guffon, P. Journeau, A. Brassier, J. Leger, B. Chevallier, Growth impairment and limited range of joint motion in children should raise suspicion of an attenuated form of mucopolysaccharidosis: expert opinion Eur J Pediatr 178 (2019) 593-603.
[12] S. Mitrovic, H. Gouze, L. Gossec, T. Schaeyerbeke, B. Fautrel, Mucopolysaccharidoses seen in adults in rheumatology Joint Bone Spine 84 (2017) 663-670.
[13] C. M. Simonaro, M. D'Angelo, M. E. Haskins, E. H. Schuchman, Joint and bone disease in mucopolysaccharidoses VI and VII: identification of new therapeutic targets and biomarkers using animal models Pediatr Res 57 (2005) 701-707.
[14] C. M. Simonaro, M. D'Angelo, X. He, E. Eliyahu, N. Shtraizent, M. E. Haskins, E. H. Schuchman, Mechanism of glycosaminoglycan-mediated bone and joint disease: implications for the mucopolysaccharidoses and other connective tissue diseases Am J Pathol 172 (2008) 112-122.
[15] S. H. Peck, P. J. O'Donnell, J. L. Kang, N. R. Malhotra, G. R. Dodge, M. Pacifici, E. M. Shore, M. E. Haskins, L. J. Smith, Delayed hypertrophic differentiation of epiphyseal chondrocytes contributes to failed secondary ossification in mucopolysaccharidosis VII dogs Molecular Genetics & Metabolism 116 (2015) 195-203.
[16] C. Taylor, P. Brady, A. O'Meara, D. Moore, F. Dowling, E. Fogarty, Mobility in Hurler syndrome J Pediatr Orthop 28 (2008) 163-168.
[17] E. J. Langereis, M. M. den Os, C. Breen, S. A. Jones, O. C. Knaven, J. Mercer, W. P. Miller, P. M. Kelly, J. Kennedy, T. G. Ketterl, A. O'Meara, P. J. Orchard, T. C. Lund, R. R. van Rijn, R. J. Sakkers, K. K. White, F. A. Wijburg, Progression of Hip Dysplasia in Mucopolysaccharidosis Type I Hurler After Successful Hematopoietic Stem Cell Transplantation J Bone Joint Surg Am 98 (2016) 386-395.
[18] H. C. Lao, Y. C. Lin, M. L. Liang, Y. W. Yang, Y. H. Huang, Y. L. Chan, Y. W. Hsu, S. P. Lin, C. K. Chuang, J. K. Cheng, H. Y. Lin, The Anesthetic Strategy for Patients with Mucopolysaccharidoses: A Retrospective Cohort Study J Pers Med 12 (2022).
[19] J. V. Alvarez, S. B. Bravo, M. P. Chantada-Vazquez, S. Barbosa-Gouveia, C. Colon, O. Lopez-Suarez, S. Tomatsu, F. J. Otero-Espinar, M. L. Couce, Plasma Proteomic Analysis in Morquio A Disease Int J Mol Sci 22 (2021).
[20] V. J. Alvarez, S. B. Bravo, M. P. Chantada-Vazquez, C. Colon, M. J. De Castro, M. Morales, I. Vitoria, S. Tomatsu, F. J. Otero-Espinar, M. L. Couce, Characterization of New Proteomic Biomarker Candidates in Mucopolysaccharidosis Type IVA Int J Mol Sci 22 (2020).
[21] W. E. Heywood, S. Camuzeaux, I. Doykov, N. Patel, R. L. Preece, E. Footitt, M. Cleary, P. Clayton, S. Grunewald, L. Abulhoul, A. Chakrapani, N. J. Sebire, P. Hindmarsh, T. J. de Koning, S. Heales, D. Burke, P. Gissen, K. Mills, Proteomic Discovery and Development of a Multiplexed Targeted MRM-LC-MS/MS Assay for Urine Biomarkers of Extracellular Matrix Disruption in Mucopolysaccharidoses I, II, and VI Anal Chem 87 (2015) 12238-12244.
[22] X. Yuan, Y. Meng, C. Chen, S. Liang, Y. Ma, W. Jiang, J. Duan, C. Wang, Proteomic approaches in the discovery of potential urinary biomarkers of mucopolysaccharidosis type II Clin Chim Acta 499 (2019) 34-40.
[23] T. C. Lund, T. M. Doherty, J. B. Eisengart, R. L. Freese, K. D. Rudser, E. B. Fung, B. S. Miller, K. K. White, P. J. Orchard, C. B. Whitley, L. E. Polgreen, Biomarkers for prediction of skeletal disease progression in mucopolysaccharidosis type I JIMD Rep 58 (2021) 89-99.
[24] K. P. Menon, P. T. Tieu, E. F. Neufeld, Architecture of the canine IDUA gene and mutation underlying canine mucopolysaccharidosis I Genomics 14 (1992) 763-768.
[25] R. M. Shull, R. G. Helman, E. Spellacy, G. Constantopoulos, R. J. Munger, E. F. Neufeld, Morphologic and biochemical studies of canine mucopolysaccharidosis I The American journal of pathology 114 (1984) 487-495.
[26] E. Spellacy, R. M. Shull, G. Constantopoulos, E. F. Neufeld, A canine model of human alpha-L-iduronidase deficiency Proc Natl Acad Sci USA 80 (1983) 6091-6095.
[27] R. Bruderer, O. M. Bernhardt, T. Gandhi, S. M. Miladinovic, L. Y. Cheng, S. Messner, T. Ehrenberger, V. Zanotelli, Y. Butscheid, C. Escher, O. Vitek, O. Rinner, L. Reiter, Extending the limits of quantitative proteome profiling with data-independent acquisition and application to acetaminophen-treated three-dimensional liver microtissues Mol Cell Proteomics 14 (2015) 1400-1410.
[28] S. Tyanova, T. Temu, P. Sinitcyn, A. Carlson, M. Y. Hein, T. Geiger, M. Mann, J. Cox, The Perseus computational platform for comprehensive analysis of (prote) omics data Nat Methods 13 (2016) 731-740.
[29] V. Galindo-Zamora, P. Dziallas, D. C. Ludwig, I. Nolte, P. Wefstaedt, Diagnostic accuracy of a short-duration 3 Tesla magnetic resonance protocol for diagnosing stifle joint lesions in dogs with non-traumatic cranial cruciate ligament rupture BMC Vet Res 9 (2013) 40.
[30] P. R. Kornaat, R. Y. Ceulemans, H. M. Kroon, N. Riyazi, M. Kloppenburg, W. O. Carter, T. G. Woodworth, J. L. Bloem, MRI assessment of knee osteoarthritis: Knee Osteoarthritis Scoring System (KOSS) —inter-observer and intra-observer reproducibility of a compartment-based scoring system Skeletal Radiol 34 (2005) 95-102.
[31] D. J. Hunter, A. Guermazi, G. H. Lo, A. J. Grainger, P. G. Conaghan, R. M. Boudreau, F. W. Roemer, Evolution of semi-quantitative whole joint assessment of knee OA: MOAKS (MRI Osteoarthritis Knee Score) Osteoarthritis and cartilage 19 (2011) 990-1002.
[32] J. L. Cook, K. Kuroki, D. Visco, J. P. Pelletier, L. Schulz, F. P. Lafeber, The OARSI histopathology initiative-recommendations for histological assessments of osteoarthritis in the dog Osteoarthritis Cartilage 18 Suppl 3 (2010) S66-79.
[33] C. L. Hill, D. G. Gale, C. E. Chaisson, K. Skinner, L. Kazis, M. E. Gale, D. T. Felson, Knee effusions, popliteal cysts, and synovial thickening: association with knee pain in osteoarthritis J Rheumatol 28 (2001) 1330-1337.
[34] Y. Fujii, L. Liu, L. Yagasaki, M. Inotsume, T. Chiba, H. Asahara, Cartilage Homeostasis and Osteoarthritis Int J Mol Sci 23 (2022).
[35] R. Gawri, Y. K. Lau, G. Lin, S. S. Shetye, C. Zhang, Z. Jiang, K. Abdoun, C. R. Scanzello, S. Y. Jo, W. Mai, G. R. Dodge, M. L. Casal, L. J. Smith, Dose-Dependent Effects of Enzyme Replacement Therapy on Skeletal Disease Progression in Mucopolysaccharidosis VII Dogs Molecular Therapy-Methods and Clinical Development In press (2022).
[36] C. M. Simonaro, Y. Ge, E. Eliyahu, X. He, K. J. Jepsen, E. H. Schuchman, Involvement of the Toll-like receptor 4 pathway and use of TNF-alpha antagonists for treatment of the mucopolysaccharidoses Proc Natl Acad Sci USA 107 (2010) 222-227.
[37] P. G. de Oliveira, G. Baldo, F. Q. Mayer, B. Martinelli, L. Meurer, R. Giugliani, U. Matte, R. M. Xavier, Characterization of joint disease in mucopolysaccharidosis type I mice Int J Exp Pathol 94 (2013) 305-311.
[38] L. E. Polgreen, R. K. Vehe, K. Rudser, A. Kunin-Batson, J. J. Utz, P. Dickson, E. Shapiro, C. B. Whitley, Elevated TNF-alpha is associated with pain and physical disability in mucopolysaccharidosis types I, II, and VI Mol Genet Metab 117 (2016) 427-430.
[39] N. Y. Ferreira, C. C. do Nascimento, V. G. Pereira, F. de Oliveira, C. C. Medalha, V. C. da Silva, V. D'Almeida, Biomechanical and histological characterization of MPS I mice femurs Acta Histochem 123 (2021) 151678.
[40] J. E. Pope, E. H. Choy, C-reactive protein and implications in rheumatoid arthritis and associated comorbidities Semin Arthritis Rheum 51 (2021) 219-229.
[41] X. Jin, J. R. Beguerie, W. Zhang, L. Blizzard, P. Otahal, G. Jones, C. Ding, Circulating C reactive protein in osteoarthritis: a systematic review and meta-analysis Ann Rheum Dis 74 (2015) 703-710.
[42] R. Gomez, J. Conde, M. Scotece, J. J. Gomez-Reino, F. Lago, O. Gualillo, What's new in our understanding of the role of adipokines in rheumatic diseases? Nat Rev Rheumatol 7 (2011) 528-536.
[43] F. Gonzalez-Ponce, J. I. Gamez-Nava, E. E. Perez-Guerrero, A. M. Saldana-Cruz, M. L. Vazquez-Villegas, J. M. Ponce-Guarneros, M. Huerta, X. Trujillo, B. Contreras-Haro, A. D. Rocha-Munoz, M. O. Carrillo-Escalante, E. N. Sanchez-Rodriguez, E. E. Gomez-Ramirez, C. A. Nava-Valdivia, E. G. Cardona-Munoz, L. Gonzalez-Lopez, D. Research Group for the Assessment of Prognosis Biomarkers in Autoimmune, Serum chemerin levels: A potential biomarker of joint inflammation in women with rheumatoid arthritis PLOS One 16 (2021) e0255854.
[44] K. Huang, G. Du, L. Li, H. Liang, B. Zhang, Association of chemerin levels in synovial fluid with the severity of knee osteoarthritis Biomarkers 17 (2012) 16-20.
[45] U. T. Timur, H. Jahr, J. Anderson, D. C. Green, P. J. Emans, A. Smagul, L. W. van Rhijn, M. J. Peffers, T. J. M. Welting, Identification of tissue-dependent proteins in knee OA synovial fluid Osteoarthritis Cartilage 29 (2021) 124-133.
[46] J. J. Biskup, D. G. Balogh, K. H. Haynes, A. L. Freeman, M. G. Conzemius, Mechanical strength of four allograft fixation techniques for ruptured cranial cruciate ligament repair in dogs Am J Vet Res 76 (2015) 411-419.
[47] J. O. Stracke, A. J. Fosang, K. Last, F. A. Mercuri, A. M. Pendas, E. Llano, R. Perris, P. E. Di Cesare, G. Murphy, V. Knauper, Matrix metalloproteinases 19 and 20 cleave aggrecan and cartilage oligomeric matrix protein (COMP) FEBS Lett 478 (2000) 52-56.
[48] J. O. Stracke, M. Hutton, M. Stewart, A. M. Pendas, B. Smith, C. Lopez-Otin, G. Murphy, V. Knauper, Biochemical characterization of the catalytic domain of human matrix metalloproteinase 19. Evidence for a role as a potent basement membrane degrading enzyme J Biol Chem 275 (2000) 14809-14816.
[49] R. Sedlacek, S. Mauch, B. Kolb, C. Schatzlein, H. Eibel, H. H. Peter, J. Schmitt, U. Krawinkel, Matrix metalloproteinase MMP-19 (RASI-1) is expressed on the surface of activated peripheral blood mononuclear cells and is detected as an autoantigen in rheumatoid arthritis Immunobiology 198 (1998) 408-423.
[50] F. Bost, M. Diarra-Mehrpour, J. P. Martin, Inter-alpha-trypsin inhibitor proteoglycan family—a group of proteins binding and stabilizing the extracellular matrix Eur J Biochem 252 (1998) 339-346.
[51] L. Zhuo, K. Kimata, Structure and function of inter-alpha-trypsin inhibitor heavy chains Connect Tissue Res 49 (2008) 311-320.
[52] J. Sandson, D. Hamerman, G. Schwick, Altered properties of pathological hyaluronate due to a bound inter-alpha trypsin inhibitor Trans Assoc Am Physicians 78 (1965) 304-313.
[53] S. M. Smith, J. Melrose, A Retrospective Analysis of the Cartilage Kunitz Protease Inhibitory Proteins Identifies These as Members of the Inter-alpha-Trypsin Inhibitor Superfamily with Potential Roles in the Protection of the Articulatory Surface Int J Mol Sci 20 (2019).
[54] B. Akerstrom, M. Gram, A1M, an extravascular tissue cleaning and housekeeping protein Free Radic Biol Med 74 (2014) 274-282.
[55] S. Larsson, B. Akerstrom, M. Gram, L. S. Lohmander, A. Struglics, alpha1-Microglobulin Protects Against Bleeding-Induced Oxidative Damage in Knee Arthropathies Front Physiol 9 (2018) 1596.
[56] K. Pierzynowska, L. Gaffke, Z. Cyske, G. Wegrzyn, B. Buttari, E. Profumo, L. Saso, Oxidative Stress in Mucopolysaccharidoses: Pharmacological Implications Molecules 26 (2021).
[57] C. M. Simonaro, M. E. Haskins, E. H. Schuchman, Articular chondrocytes from animals with a dermatan sulfate storage disease undergo a high rate of apoptosis and release nitric oxide and inflammatory cytokines: a possible mechanism underlying degenerative joint disease in the mucopolysaccharidoses Lab Invest 81 (2001) 1319-1328.
[58] C. Karlsson, T. Dehne, A. Lindahl, M. Brittberg, A. Pruss, M. Sittinger, J. Ringe, Genome-wide expression profiling reveals new candidate genes associated with osteoarthritis Osteoarthr Cartilage 18 (2010) 581-592.
[59] M. Singh, F. Del Carpio-Cano, J. Y. Belcher, K. Crawford, N. Frara, T. A. Owen, S. N. Popoff, F. F. Safadi, Functional Roles of Osteoactivin in Normal and Disease Processes Crit Rev Eukar Gene 20 (2010) 341-357.
[60] N. Frara, S. M. Abdelmagid, G. R. Sondag, F. M. Moussa, V. R. Yingling, T. A. Owen, S. N. Popoff, M. F. Barbe, F. F. Safadi, Transgenic Expression of Osteoactivin/gpnmb Enhances Bone Formation In Vivo and Osteoprogenitor Differentiation Ex Vivo J Cell Physiol 231 (2016) 72-83.
[61] G. Baldo, S. S. Wu, R. A. Howe, M. Ramamoothy, R. H. Knutsen, J. L. Fang, R. P. Mecham, Y. L. Liu, X. B. Wu, J. P. Atkinson, K. P. Ponder, Pathogenesis of aortic dilatation in mucopolysaccharidosis VII mice may involve complement activation Molecular Genetics and Metabolism 104 (2011) 608-619.
[62] M. K. Parente, R. Rozen, C. N. Cearley, J. H. Wolfe, Dysregulation of Gene Expression in a Lysosomal Storage Disease Varies between Brain Regions Implicating Unexpected Mechanisms of Neuropathology Plos One 7 (2012).
[63] G. Kramer, W. Wegdam, W. Donker-Koopman, R. Ottenhoff, P. Gaspar, M. Verhoek, J. Nelson, T. Gabriel, W. Kallemeijn, R. G. Boot, J. D. Laman, J. P. C. Vissers, T. Cox, E. Pavlova, M. T. Moran, J. M. Aerts, M. van Eijk, Elevation of glycoprotein nonmetastatic melanoma protein B in type 1 Gaucher disease patients and mouse models Febs Open Bio 6 (2016) 902-913.
[64] V. Murugesan, J. Liu, R. H. Yang, H. Q. Lin, A. Lischuk, G. Pastores, X. K. Zhang, W. L. Chuang, P. K. Mistry, Validating glycoprotein non-metastatic melanoma B (gpNMB, osteoactivin), a new biomarker of Gaucher disease Blood Cell Mol Dis 68 (2018) 47-53.
[65] H. Zigdon, A. Savidor, Y. Levin, A. Meshcheriakova, R. Schiffmann, A. H. Futerman, Identification of a Biomarker in Cerebrospinal Fluid for Neuronopathic Forms of Gaucher Disease Plos One 10 (2015).
[66] S. H. Peck, J. W. Tobias, E. M. Shore, N. R. Malhotra, M. E. Haskins, M. L. Casal, L. J. Smith, Molecular profiling of failed endochondral ossification in mucopolysaccharidosis VII Bone 128 (2019) 115042.
[67] N. Schafer, S. Grassel, Involvement of complement peptides C3a and C5a in osteoarthritis pathology Peptides 154 (2022) 170815.
[68] V. M. Holers, N. K. Banda, Complement in the Initiation and Evolution of Rheumatoid Arthritis Front Immunol 9 (2018) 1057.
[69] G. Baldo, S. Wu, R. A. Howe, M. Ramamoothy, R. H. Knutsen, J. Fang, R. P. Mecham, Y. Liu, X. Wu, J. P. Atkinson, K. P. Ponder, Pathogenesis of aortic dilatation in mucopolysaccharidosis VII mice may involve complement activation Mol Genet Metab 104 (2011) 608-619.
[70] M. Galindo-Izquierdo, J. L. Pablos Alvarez, Complement as a Therapeutic Target in Systemic Autoimmune Diseases Cells 10 (2021).
[71] Y. Perez-Riverol, J. Bai, C. Bandla, D. Garcia-Seisdedos, S. Hewapathirana, S. Kamatchinathan, D. J. Kundu, A. Prakash, A. Frericks-Zipper, M. Eisenacher, M. Walzer, S. Wang, A. Brazma, J. A. Vizcaino, The PRIDE database resources in 2022: a hub for mass spectrometry-based proteomics evidences Nucleic Acids Res 50 (2022) D543-D552.

TABLE 4
Molecules that exhibited significant correlation
in abundance between synovial fluid and serum

| | | Synovial | | Pearson | |
|---|---|---|---|---|---|
| | | Fluid | Serum | Correlation | |
| Symbol | Full Name | log2FC | log2FC | r value | p value |
| A1M | Alpha-1-microglobulin | 1.36 | 0.78 | 0.82 | 0.002 |
| AMY | Alpha-amylase | 1.22 | 1.05 | 0.85 | 0.001 |
| ANGPTL3 | Angiopoietin like 3 | 1.21 | 0.60 | 0.82 | 0.002 |
| AOC3 | Amine Oxidase Copper Containing | −1.02 | −1.36 | 0.87 | 0.001 |
| | 3 | | | | |
| APBB1 | Amyloid Beta Precursor Protein | 1.15 | 0.78 | 0.80 | 0.003 |
| | Binding Family B Member 1 | | | | |
| APOH | Beta-2-glycoprotein 1 | 0.63 | 0.38 | 0.70 | 0.016 |
| ATRN | Attractin | 0.73 | 0.66 | 0.75 | 0.008 |
| AZGP1 | Alpha-2-glycoprotein 1, zinc- | 1.14 | 0.99 | 0.86 | 0.001 |
| | binding | | | | |
| B2M | Beta-2-microglobulin | 0.93 | 1.01 | 0.69 | 0.020 |
| C2 | Complement C2 | 0.86 | 0.53 | 0.81 | 0.002 |
| C4A | C4a anaphylatoxin | 0.97 | 0.64 | 0.86 | 0.001 |
| C9 | Complement C9 | 0.79 | 0.84 | 0.73 | 0.011 |
| CANT1 | Calcium Activated Nucleotidase 1 | 0.83 | 1.50 | 0.80 | 0.003 |
| CD86 | CD86 molecule | 1.56 | 1.59 | 0.72 | 0.012 |
| CDH1 | Cadherin 1-like | 2.22 | 1.75 | 0.96 | <0.001 |
| CPN1 | Carboxypeptidase N subunit 1 | 1.23 | 0.98 | 0.86 | 0.001 |
| CPN2 | Carboxypeptidase N subunit 2 | 1.04 | 1.00 | 0.91 | <0.001 |
| CREB3L3 | cAMP responsive element binding | 2.29 | 2.26 | 0.87 | 0.005 |
| | protein 3 like 3 | | | | |
| CRP | C-reactive protein | 2.12 | 2.89 | 0.90 | <0.001 |
| EIF4A2 | Eukaryotic Translation Initiation | −1.16 | −1.08 | 0.77 | 0.006 |
| | Factor 4A2 | | | | |
| GANAB | Glucosidase II alpha subunit | 0.75 | 1.97 | 0.64 | 0.033 |
| ITIH2 | Inter-alpha-trypsin inhibitor heavy | 0.62 | 0.46 | 0.75 | 0.008 |
| | chain 2 | | | | |
| ITIH3 | Inter-alpha-trypsin inhibitor heavy | 1.99 | 1.91 | 0.97 | <0.001 |
| | chain 3 | | | | |
| LAMP2 | Lysosomal Associated Membrane | 2.37 | 0.98 | 0.65 | 0.029 |
| | Protein 2 | | | | |
| LBP | Lipopolysaccharide-binding protein | 1.21 | 1.14 | 0.90 | <0.001 |
| LRG1 | Leucine rich alpha-2-glycoprotein 1 | 0.80 | 0.71 | 0.74 | 0.009 |
| MAN2B1 | Alpha-mannosidase | 1.84 | 1.67 | 0.94 | <0.001 |
| MGAM | Maltase-glucoamylase | −0.84 | −1.07 | 0.88 | <0.001 |
| MMP19 | Matrix metalloproteinase 19 | 3.35 | 1.29 | 0.88 | <0.001 |
| ORM1 | Orosomucoid | 1.47 | 1.21 | 0.88 | <0.001 |
| PSAP | Prosaposin | 1.45 | 1.35 | 0.94 | <0.001 |
| PTPRG | Protein-tyrosine-phosphatase | 0.81 | 0.70 | 0.77 | 0.006 |
| | Receptor Type G | | | | |
| QSOX1 | Quiescin Sulfhydryl Oxidase 1 | 1.09 | 0.96 | 0.90 | <0.001 |
| RARRES2 | Chemerin | 1.50 | 1.23 | 0.82 | 0.002 |
| SERPINA3 | SERPIN domain-containing protein | 0.71 | 0.34 | 0.61 | 0.045 |
| SERPINA7 | Serpin family A member 7 | 0.71 | 1.04 | 0.94 | <0.001 |
| SPP2 | Secreted phosphoprotein 2 | 1.62 | 1.23 | 0.83 | 0.002 |
| TFRC | Transferrin receptor protein 1 | −0.74 | −0.91 | 0.84 | 0.001 |
| URAH | 5-hydroxyisourate hydrolase | 1.68 | 1.48 | 0.98 | <0.001 |
| VSIG4 | V-set and immunoglobulin domain | 1.76 | 1.77 | 0.70 | 0.017 |
| | containing 4 | | | | |

### Detailed Proteomics Methodology

Serum and synovial fluid samples were thawed, centrifuged at 20,000 g for 10 minutes, and total protein concentration was determined using the bicinchoninic acid assay. Synovial fluid was pretreated with hyaluronidase (1 ng/μl at 37° C. under agitation for 2 hours; H3884; Sigma Aldrich; St Louis, USA) following established techniques.¹ Proteins in both synovial fluid and serum were then solubilized and digested with the iST kit (PreOmics GmbH; Martinsried, Germany) as per the manufacturer's protocol. Briefly, the samples (1 ul of serum or hyaluronidase-treated synovial fluid) were solubilized, reduced and alkylated by addition of sodium deoxycholate buffer containing tris(2-carboxyethyl)phosphine and 2-chloroacetamide then heated to 95° C. for 10 minutes. Proteins were then enzymatically hydrolyzed for 1.5 hours at 37° C. by addition of LysC and trypsin (Promega, Fujifilm Wako chemicals, USA). The resulting peptides were de-salted, dried by vacuum centrifugation, and reconstituted in 0.1% trifluoroacetic acid containing iRT peptides (Biognosys Schlieren, Switzerland).

For mass spectrometry (MS), samples were randomized and 2 μg of each was analyzed on a QExactive HF mass spectrometer coupled with an Ultimate 3000 nano ultra-performance liquid chromatography system and an EasySpray source (Thermofisher Scientific; San Jose, CA). Peptides were loaded onto an 75 μm×2 cm trap column (Acclaim PepMap 100; Thermofisher) at 5 μl/min, and separated by reverse phase high performance liquid chromatography on a 75 μm internal diameter×50 cm 2 μm PepMap rapid separation liquid chromatography C18 column (Thermofisher). Mobile phase A consisted of 0.1% formic acid and mobile phase B of 0.1% formic acid/acetonitrile. The gradient was started at 1% phase B from 0-3 minutes, 5% phase B at 5 minutes, 15% B at 15 minutes, 45% at 155 minutes before increasing to 99% B to wash column off. Flow rate started at 300 nL/min, was lowered to 210 nL/min from 15.1 to 155 minutes and increased back to 300 nL/min. Data was acquired using data independent acquisition (DIA). Mass spectrometer settings were: one full MS scan at 120,000 resolution and a scan range of 300-1650 m/z with an automatic gain control (AGC) target of 3×10⁶ and a maximum inject time of 60 ms. This was followed by 22 (DIA) isolation windows with varying sizes at 30,000 resolution, an AGC target of 3×10⁶, and injection times set to auto. The default charge state was 4, the first mass was fixed at 200 m/z and the normalized collision energy for each window was stepped at 25.5, 27 and 30.

The suitability of Q Exactive HF instrument was monitored using QuiC software (Biognosys; Schlieren, Switzerland) for the analysis of the spiked-in iRT peptides. As a measure for quality control, we injected standard E. coli protein digest in between samples (one injection after every four biological samples) and collected the data in data dependent acquisition (DDA) mode. The collected DDA data were analyzed in MaxQuant² and the output was subsequently visualized using the PTXQC³ package to track the quality of the instrumentation. Statistical analysis of acquired data were performed as described below.

The raw files for DIA analysis were processed with Spectronaut⁴ version 15.1 in DirectDIA mode using reference Canine proteome from UniProt (45,351 canonical and isoform proteins). The default settings in Spectronaut were used for peptide and protein quantification. Perseus (1.6.14.0)⁵ was employed for data processing and statistical analysis using the MS2 intensity values generated by Spectronaut. The data were log 2 transformed and normalized by subtracting the median for each sample. Student's t-test with was employed to identify differentially abundant proteins using adj.p.value<0.05 as significant threshold. The proteins that were exclusively detected in one experimental group were also reported for further bioinformatics analysis.

### References for Detailed Proteomics Methodology

[1] Anderson J R, Phelan M M, Rubio-Martinez L M, Fitzgerald M M, Jones S W, Clegg P D, Peffers M J: Optimization of Synovial Fluid Collection and Processing for NMR Metabolomics and LC-MS/MS Proteomics. J Proteome Res 2020, 19:2585-97.
[2] Tyanova S, Temu T, Cox J: The MaxQuant computational platform for mass spectrometry-based shotgun proteomics. Nat Protoc 2016, 11:2301-19.
[3] Bielow C, Mastrobuoni G, Kempa S: Proteomics Quality Control: Quality Control Software for MaxQuant Results. J Proteome Res 2016, 15:777-87.
[4] Bruderer R, Bernhardt O M, Gandhi T, Miladinovic S M, Cheng L Y, Messner S, Ehrenberger T, Zanotelli V, Butscheid Y, Escher C, Vitek O, Rinner O, Reiter L: Extending the limits of quantitative proteome profiling with data-independent acquisition and application to acetaminophen-treated three-dimensional liver microtissues. Mol Cell Proteomics 2015, 14:1400-10.
[5] Tyanova S, Temu T, Sinitcyn P, Carlson A, Hein M Y, Geiger T, Mann M, Cox J: The Perseus computational platform for comprehensive analysis of (prote) omics data. Nat Methods 2016, 13:731-40.

TABLE 5
Molecules significantly upregulation in the synovial
fluid of MPS I animals compared to controls.

| | | | log2 | p |
|---|---|---|---|---|
| Genes | Accession | Full Name | FC | value |
| ADGRE5 | A0A5F4C889 | Adhesion G protein-coupled receptor E5 | 1.08 | <0.001 |
| A1M | J9NUI6 | Alpha-1-microglobulin | 1.36 | <0.001 |
| AMYA | A0A5F4BUM0 | Alpha-amylase | 1.22 | <0.001 |
| ANGPTL3 | E2QZ98 | Angiopoietin like 3 | 1.21 | 0.001 |
| APBB1 | F1PZR4 | Amyloid beta precursor protein binding | 1.15 | <0.001 |
| | | family B member 1 | | |
| APOE | A0A5S7EUL7 | Apolipoprotein E | 2.22 | <0.001 |
| APOH | P33703 | Beta-2-glycoprotein 1 | 0.63 | 0.012 |
| ATP6AP1 | A0A5F4CCB2 | ATPase H+ transporting accessory protein | 2.11 | <0.001 |
| | | 1 | | |
| ATP6AP2 | A0A5F4BQQ2 | ATPase H(+)-transporting lysosomal | 0.81 | 0.006 |
| | | accessory protein 2 | | |
| ATRN | A0A5F4DKK2 | Attractin | 0.73 | 0.005 |
| AZGP1 | F1PDJ7 | Alpha-2-glycoprotein 1, zinc-binding | 1.14 | <0.001 |
| B2M | A0A5F4C4Q1 | Beta-2-microglobulin | 0.93 | 0.003 |
| BPI | A0A5F4CJ11 | Bactericidal permeability-increasing | 1.98 | 0.009 |
| | | protein | | |
| C2 | A0A5F4CR45 | C3/C5 convertase | 0.86 | 0.004 |
| C4A | A0A5F4D5S2 | C4a anaphylatoxin | 0.97 | <0.001 |
| C9 | E2RFV9 | Complement C9 | 0.79 | 0.007 |
| CANTI | A0A5F4CPH1 | Galectin-3-binding protein | 0.83 | <0.001 |
| CD86 | A0A5F4DDS5 | CD86 molecule | 1.56 | <0.001 |
| CD93 | F1PCS8 | CD93 molecule | 0.61 | 0.011 |
| CDH1 | F1PAB0 | Cadherin-like 1 | 2.22 | <0.001 |
| CHI3L1 | A0A5F4CYU5 | Chitinase-3-like protein 1 | 4.87 | <0.001 |
| CLE1 | A0A5F4DC46 | Endostatin domain-containing protein | 0.91 | 0.006 |
| COL1A1 | A0A5F4CZS5 | Collagen alpha-1(I) chain | 1.47 | 0.001 |
| COL6A3 | F1PR31 | Collagen type VI alpha 3 chain | 0.89 | 0.014 |
| CORO1A | A0A5F4CI56 | Coronin | 1.11 | 0.005 |
| CPN1 | E2RNG3 | Carboxypeptidase N subunit 1 | 1.23 | <0.001 |
| CPN2 | J9PAA4 | Carboxypeptidase N subunit 2 | 1.04 | 0.001 |
| CPQ | A0A5F4BXJ5 | Carboxypeptidase Q | 0.76 | 0.006 |
| CREB3L3 | F1Q1J8 | cAMP responsive element binding protein | 2.29 | 0.001 |
| | | 3 like 3 | | |
| CRP | E2R5J0 | Pentaxin | 2.12 | 0.003 |
| CSF1R | A0A5F4CGY4 | Receptor protein-tyrosine kinase | 1.09 | 0.003 |
| CTSB | A0A5F4CYW8 | Cathepsin B | 1.48 | <0.001 |
| CTSC | F1PSK8 | Cathepsin C | 1.31 | 0.001 |
| CTSD | Q4LAL9 | Cathepsin D | 2.72 | <0.001 |
| CTSH | F6X9C1 | Cathepsin H | 1.28 | 0.006 |
| CTSS | F1PAK0 | Cathepsin S | 0.58 | 0.004 |
| CTSZ | A0A5F4C1H3 | Cathepsin X | 2.12 | <0.001 |
| DNASE2 | J9JHU4 | Deoxyribonuclease II | 3.80 | <0.001 |
| F10 | A0A5F4CM58 | Coagulation factor X | 0.79 | 0.009 |
| FGB | F1PGS2 | Fibrinogen beta chain | 1.29 | 0.010 |
| FGG | F1P8G0 | Fibrinogen gamma chain | 1.50 | 0.001 |
| FSTL1 | F1PY69 | Follistatin like 1 | 0.65 | 0.016 |
| GANAB | A0A5F4CVB8 | Glucosidase II alpha subunit | 0.75 | 0.006 |
| GGH | A0A5F4CXI0 | Folate gamma-glutamyl hydrolase | 3.81 | <0.001 |
| GPNMB | E2QUR6 | Glycoprotein nmb | 6.32 | <0.001 |
| GUSB | A0A5F4C573 | Beta-glucuronidase | 2.03 | 0.002 |
| HAVCR1 | J9NVI1 | Uncharacterized protein | 1.87 | 0.009 |
| HEXA | A0A5F4CGG9 | Beta-hexosaminidase | 3.79 | 0.000 |
| HEXB | F1Q1M8 | Beta-N-acetylhexosaminidase | 2.03 | 0.001 |
| HMGB2 | E2QY30 | High mobility group box 2 | 3.80 | 0.008 |
| HNRNPF | E2RB90 | Heterogeneous nuclear ribonucleoprotein | 0.50 | 0.003 |
| | | F | | |
| IGHE | F1PP29 | Immunoglobulin heavy constant epsilon | 3.60 | 0.014 |
| ITIH2 | F1PG39 | Inter-alpha-trypsin inhibitor heavy chain 2 | 0.62 | 0.007 |
| ITIH3 | H9GWG4 | Inter-alpha-trypsin inhibitor heavy chain 3 | 1.99 | <0.001 |
| LAMP1 | F1Q260 | Lysosomal associated membrane protein | 0.94 | 0.007 |
| | | 1 | | |
| LAMP2 | A0A5F4BQK3 | Lysosomal associated membrane protein | 2.37 | <0.001 |
| | | 2 | | |
| LBP | E2QW61 | Lipopolysaccharide-binding protein | 1.21 | 0.014 |
| LCN2 | E2RSM4 | Lipocalin 2 | 1.67 | 0.002 |
| LCP1 | A0A5F4D8N3 | Lymphocyte cytosolic protein 1 | 0.74 | 0.002 |
| LGALS3 | F6PME1 | Galectin | 0.73 | <0.001 |
| LMAN2 | A0A5S6CZU7 | Vesicular integral-membrane protein | 1.02 | <0.001 |
| | | VIP36 | | |
| LRG1 | E2R833 | Leucine rich alpha-2-glycoprotein 1 | 0.80 | <0.001 |
| LYZ | P81709 | Lysozyme C, spleen isozyme | 1.33 | <0.001 |
| MAN2B1 | F1PKB2 | Alpha-mannosidase | 1.84 | <0.001 |
| MARCKS | A0A5F4DDG2 | Uncharacterized protein | 0.82 | 0.002 |
| MMP19 | E2QW82 | Matrix metalloproteinase 19 | 3.35 | <0.001 |
| N/A | F1PVR5 | Uncharacterized protein | 1.71 | <0.001 |
| NIBAN2 | E2RR68 | Niban apoptosis regulator 2 | 1.20 | 0.007 |
| ORM | F6Y713 | Orosomucoid | 1.47 | 0.006 |
| PLA2G7 | A0A5F4CWA2 | Platelet-activating factor acetylhydrolase | 0.81 | 0.012 |
| PLD3 | E2QVP0 | Phospholipase D family member 3 | 2.66 | <0.001 |
| PSAP | E2RLF1 | Prosaposin | 1.45 | <0.001 |
| PSMB9 | Q5W412 | Proteasome subunit beta | 1.30 | 0.002 |
| PSME2 | A0A5F4C800 | Proteasome activator subunit 2 | 0.64 | 0.016 |
| PTGDS | A0A5F4C7K0 | Prostaglandin-H2 D-isomerase | 1.62 | 0.003 |
| PTPN6 | A0A5F4CI19 | Tyrosine-protein phosphatase non- | 1.38 | 0.003 |
| | | receptor type | | |
| PTPRG | A0A5F4BRR6 | Protein-tyrosine-phosphatase | 0.81 | 0.002 |
| QSOX1 | A0A5F4CZM1 | Sulfhydryl oxidase | 1.09 | <0.001 |
| RAC2 | E2RJH1 | Rac family small GTPase 2 | 1.82 | 0.002 |
| RARRES2 | A0A5F4DC08 | Chemerin | 1.50 | <0.001 |
| RNASE4 | A0A5F4DEH1 | RNAse Pc domain-containing protein | 1.04 | <0.001 |
| RNASET2 | A0A5F4BYC2 | Uncharacterized protein | 2.04 | <0.001 |
| SCG3 | A0A5F4BRM1 | Secretogranin III | 1.22 | 0.001 |
| SCPEP1 | F1PSP6 | Carboxypeptidase | 2.05 | 0.001 |
| SERPINA3 | F1PH87 | SERPIN domain-containing protein | 0.71 | 0.014 |
| SERPINA7 | A0A5F4D9Z3 | Serpin family A member 7 | 1.18 | 0.004 |
| SERPING1 | A0A5F4C5C9 | Serpin family G member 1 | 0.54 | 0.007 |
| SMPDL3A | J9NYQ3 | Acid sphingomyelinase-like | 0.94 | 0.005 |
| | | phosphodiesterase | | |
| SPP1 | A0A5F4BRW4 | Secreted phosphoprotein 1 | 1.37 | 0.008 |
| SPP2 | F1PD34 | Secreted phosphoprotein 2 | 1.62 | 0.002 |
| SSB | E2RH09 | Small RNA binding exonuclease | 1.20 | 0.011 |
| | | protection factor La | | |
| TGFBR3 | F1PIG0 | Transforming growth factor beta receptor | 0.90 | 0.011 |
| | | 3 | | |
| TGOLN2 | J9NYN4 | Uncharacterized protein | 1.28 | <0.001 |
| THBS3 | A0A5F4BNY8 | Thrombospondin 3 | 1.38 | <0.001 |
| TLN1 | F1PSC2 | Talin 1 | 0.68 | <0.001 |
| URAH | E2R5E9 | 5-hydroxyisourate hydrolase | 1.68 | 0.002 |
| VASN | E2RRN8 | Vasorin | 0.85 | 0.004 |
| VAT1 | F1PAG5 | Vesicle amine transport 1 | 1.22 | 0.001 |
| VCAM1 | F1PCD0 | Vascular cell adhesion protein 1 | 0.90 | 0.009 |
| VSIG4 | A0A5F4CA66 | V-set and immunoglobulin domain | 1.76 | <0.001 |
| | | containing 4 | | |
| WARS1 | E2RGG9 | T1-TrpRS | 0.88 | 0.012 |

TABLE 6
Molecules significantly downregulated in synovial
fluid from MPS I animals compared to controls.

| | | | log2 | p |
|---|---|---|---|---|
| Symbol | Accession | Full Name | FC | value |
| ACAN | A0A5F4DFW2 | Aggrecan core protein | −2.31 | 0.003 |
| AFP | F2Z4Q6 | Alpha fetoprotein | −0.93 | 0.008 |
| AGRN | A0A5F4C2A2 | Agrin | −1.49 | 0.009 |
| ANXA1 | F1P6B7 | Annexin | −2.53 | 0.001 |
| ANXA2 | Q6TEQ7 | Annexin A2 | −1.98 | 0.002 |
| ANXA5 | A0A5F4CYM9 | Annexin A5 | −1.95 | 0.001 |
| ANXA6 | A0A5F4D584 | Annexin A6 | −1.47 | 0.013 |
| AOC3 | A0A5F4C6Q3 | Amine oxidase | −1.02 | 0.001 |
| BGN | G1K2D8 | Biglycan | −1.68 | 0.000 |
| CA1 | F1PBK6 | Carbonic anhydrase 1 | −1.80 | <0.001 |
| CA2 | A0A5F4CJB2 | Carbonic anhydrase 2 | −2.23 | <0.001 |
| CDH13 | A0A5F4BRH6 | Cadherin-13 | −0.88 | 0.006 |
| CHAD | E2QRS3 | Chondroadherin | −2.60 | 0.006 |
| CILP | A0A5F4C9T3 | Cartilage intermediate layer protein | −2.06 | 0.002 |
| CILP2 | E2RCP2 | Cartilage intermediate layer protein 2 | −1.29 | 0.010 |
| CLEC11A | F6X6A0 | C-type lectin domain containing 11A | −2.27 | 0.004 |
| CLEC3A | E2RQC0 | C-type lectin domain family 3 member | −1.91 | 0.005 |
| | | A | | |
| DCN | Q29393 | Decorin | −1.98 | <0.001 |
| EIF4A2 | A0A0B4J198 | Eukaryotic translation initiation factor | −1.16 | 0.002 |
| | | 4A2 | | |
| EMP3 | E2RAX5 | Epithelial membrane protein 3 | −1.77 | 0.008 |
| FMOD | F1PLN5 | Fibromodulin | −1.94 | 0.000 |
| GNB1 | A0A5S6DBH8 | Guanine nucleotide-binding protein | −0.58 | 0.015 |
| | | G(I)/G(S)/G(T) subunit beta-1 | | |
| HAPLN1 | J9PAT9 | Hyaluronan and proteoglycan link | −1.69 | 0.004 |
| | | protein 1 | | |
| HBA | P60529 | Hemoglobin subunit alpha | −2.34 | <0.001 |
| HBAL | A0A5F4DHT8 | hemoglobin subunit alpha-like | −2.33 | <0.001 |
| HBB | P60524 | Hemoglobin subunit beta | −2.43 | <0.001 |
| HSPB6 | F1PWM7 | Heat shock protein family B (small) | −1.97 | 0.011 |
| | | member 6 | | |
| IGFBP6 | A0A5F4CEG3 | Insulin like growth factor binding | −2.21 | 0.016 |
| | | protein 6 | | |
| LMNA | A0A5F4D968 | Lamin A/C | −2.31 | <0.001 |
| LOC609402 | A0A5F4D7C9 | GLOBIN domain-containing protein | −2.50 | 0.002 |
| MFGE8 | A0A5F4C3L7 | Milk fat globule EGF and factor V/VIII | −3.00 | <0.001 |
| | | domain containing | | |
| MGAM | E2RT38 | Maltase-glucoamylase | −0.84 | 0.005 |
| N/A | L7N0J8 | Uncharacterized protein | −0.63 | 0.011 |
| NANS | A0A5F4C4I6 | N-acetylneuraminate synthase | −0.70 | 0.014 |
| PRDX2 | F1PCG4 | Peroxiredoxin 2 | −2.21 | 0.000 |
| PRELP | A0A5F4CHL9 | Proline and arginine rich end leucine | −1.94 | 0.000 |
| | | rich repeat protein | | |
| S100A10 | E2RGM0 | Calpactin I light chain | −3.26 | <0.001 |
| SLC3A2 | F1PRC5 | Solute carrier family 3 member 2 | −0.86 | 0.010 |
| SOD1 | Q8WNN6 | Superoxide dismutase | −0.78 | 0.007 |
| SPARC | A0A5F4D2Q1 | Osteonectin | −1.84 | 0.003 |
| TFRC | F1PEN6 | Transferrin receptor protein 1 | −0.74 | 0.015 |
| THBS4 | A0A5F4CHN8 | Thrombospondin 4 | −1.10 | 0.009 |
| THY1 | J9P7X2 | Thy-1 antigen | −0.83 | 0.003 |
| TNXB | F1Q432 | Tenascin XB | −1.41 | 0.016 |
| TUBB6 | A0A5F4BPS3 | Tubulin beta 6 class V | −2.31 | 0.001 |
| VCAN | A0A5F4BVH7 | Versican | −0.91 | 0.014 |
| VIM | A0A5F4DGN4 | Vimentin | −1.73 | <0.001 |
| YWHAZ | F1PBL1 | 14_3_3 domain-containing protein | −0.51 | 0.004 |

TABLE 7
Molecules significantly upregulated in serum from MPS I dogs compared to controls.

| Symbol | Accession | Full Name | Log2 FC | p value |
|---|---|---|---|---|
| ACTC1 | A0A5F4CF09 | Actin alpha cardiac muscle 1 | 0.70 | 0.0243 |
| AGT | F1PAL5 | Angiotensin 1-10 | 0.74 | <0.001 |
| A1M | J9NUI6 | Alpha-1-microglobulin | 0.78 | 0.0015 |
| ANGPTL3 | E2QZ98 | Angiopoietin like 3 | 0.60 | 0.0041 |
| APBB1 | F1PZR4 | Amyloid beta precursor protein | 0.78 | <0.001 |
| | | binding family B member 1 | | |
| APOA2 | E2RAK7 | Apolipoprotein A-II | 0.67 | <0.001 |
| APOB | F1P8Z5 | Apolipoprotein B | 0.84 | 0.0016 |
| APOH | P33703 | Beta-2-glycoprotein 1 | 0.38 | 0.0078 |
| ATRN | A0A5F4DKK2 | Attractin | 0.66 | 0.0239 |
| AZGP1 | F1PDJ7 | Alpha-2-glycoprotein 1, zinc-binding | 0.99 | <0.001 |
| B2M | A0A5F4C4Q1 | Beta-2-microglobulin | 1.01 | 0.0037 |
| BTD | A0A5F4CL79 | Biotinidase | 0.34 | 0.0195 |
| C1S | A0A5F4BS96 | Complement C1s | 0.19 | 0.0248 |
| C2 | A0A5F4CR45 | C3/C5 convertase | 0.53 | 0.0024 |
| C3 | F1PGM1 | C3-beta-c | 0.29 | 0.0099 |
| C4BPA | F1PGM9 | Complement component 4 binding | 0.59 | 0.0094 |
| | | protein alpha | | |
| C5 | F1P7J4 | Complement C5 | 0.39 | 0.0017 |
| C9 | E2RFV9 | Complement C9 | 0.84 | 0.0016 |
| CALR | F6UYJ9 | Calreticulin | 2.62 | 0.0211 |
| CANT1 | A0A5F4CPH1 | Galectin-3-binding protein | 1.50 | 0.0012 |
| CCL16 | E2RA70 | C-C motif chemokine | 1.02 | 0.0017 |
| CD86 | A0A5F4DDS5 | CD86 molecule | 1.59 | 0.0002 |
| CD93 | F1PCS8 | CD93 molecule | 0.58 | 0.0037 |
| CDH1 | A0A5S6DAH6 | Cadherin-1 | 1.18 | 0.0364 |
| CFB | E2RS75 | C3/C5 convertase | 0.40 | 0.0025 |
| CFI | A0A5F4C7X1 | Complement factor I | 0.23 | 0.0078 |
| COL6A3 | F1PR31 | Collagen type VI alpha 3 chain | 0.66 | 0.0026 |
| CP | A0A5F4BZM5 | Ceruloplasmin | 0.27 | 0.0295 |
| CPN1 | E2RNG3 | Carboxypeptidase N subunit 1 | 0.98 | <0.001 |
| CPN2 | J9PAA4 | Carboxypeptidase N subunit 2 | 1.00 | <0.001 |
| CREB3L3 | F1Q1J8 | cAMP responsive element binding | 2.26 | <0.001 |
| | | protein 3 like 3 | | |
| CRP | E2R5J0 | C-reactive protein/pentaxin | 2.89 | 0.0027 |
| CSF1 | A0A5F4C0H1 | Colony stimulating factor 1 | 1.06 | 0.0288 |
| F2 | E2RRM2 | Prothrombin; Activation peptide | 0.33 | <0.001 |
| | | fragment 1 | | |
| FBLN1 | A0A5F4D5V1 | Fibulin-1 | 0.75 | <0.001 |
| GANAB | A0A5F4CVB8 | Glucosidase II alpha subunit | 1.97 | <0.001 |
| GC | E2R5W6 | Gc-globulin | 0.32 | 0.0097 |
| GPLD1 | A0A5F4BY85 | Glycosyl-phosphatidylinositol-specific | 0.38 | 0.0010 |
| | | phospholipase D | | |
| ITIH1 | F1Q418 | Inter-alpha-trypsin inhibitor heavy | 0.61 | 0.0017 |
| | | chain 1 | | |
| ITIH2 | F1PG39 | Inter-alpha-trypsin inhibitor heavy | 0.46 | 0.0039 |
| | | chain 2 | | |
| ITIH3 | H9GWG4 | Inter-alpha-trypsin inhibitor heavy | 1.91 | <0.001 |
| | | chain 3 | | |
| ITIH4 | H9GWY3 | Inter-alpha-trypsin inhibitor heavy | 0.53 | 0.0284 |
| | | chain 4 | | |
| KNG1 | A0A5F4C2N3 | Kininogen 1 | 0.46 | <0.001 |
| LAMP2 | A0A5F4BQK3 | Lysosomal associated membrane | 0.98 | 0.0196 |
| | | protein 2 | | |
| LBP | E2QW61 | Lipopolysaccharide-binding protein | 1.14 | 0.0207 |
| ORM1 | F6Y713 | Alpha-1-acid glycoprotein | 1.21 | 0.0052 |
| FTL | A0A5S6DQF4 | Ferritin | 1.98 | 0.0110 |
| C4a | A0A5F4D5S2 | C4a anaphylatoxin | 0.64 | 0.0013 |
| CDH1 | F1PAB0 | Cadherin 1-like | 1.75 | <0.001 |
| AMYA | A0A5F4BUM0 | Alpha-amylase | 1.05 | <0.001 |
| LOC611458 | A0A5F4C6H3 | Pregnancy zone protein-like | 0.30 | 0.0151 |
| LRG1 | E2R833 | Leucine rich alpha-2-glycoprotein 1 | 0.71 | 0.0021 |
| LRP1 | A0A5F4CYC3 | LDL receptor related protein 1 | 0.95 | 0.0021 |
| MAN2B1 | F1PKB2 | Alpha-mannosidase | 1.67 | 0.0018 |
| MASP1 | A0A5F4BQ38 | Mannan binding lectin serine | 0.71 | 0.0020 |
| | | peptidase 1 | | |
| MBL2 | J9NWG4 | C-type lectin domain-containing | 0.66 | 0.0017 |
| | | protein | | |
| MFAP4 | A0A5F4BZA4 | Microfibril associated protein 4 | 0.90 | 0.0013 |
| MMP19 | E2QW82 | Matrix metalloproteinase 19 | 1.29 | <0.001 |
| OAF | J9NRG9 | Out at first protein homolog | 0.63 | 0.0078 |
| OMD | E2RNR9 | Osteomodulin | 0.75 | 0.0071 |
| PDGFRB | F1PE35 | Platelet-derived growth factor receptor | 0.60 | 0.0082 |
| | | beta | | |
| PLG | F1Q421 | Plasminogen | 0.25 | 0.0101 |
| POSTN | A0A5F4CN27 | Periostin | 1.38 | 0.0099 |
| PSAP | E2RLF1 | Prosaposin | 1.35 | <0.001 |
| PSMB3 | E2QX17 | Proteasome subunit beta | 0.48 | 0.0320 |
| PTPRG | A0A5F4BRR6 | Protein-tyrosine-phosphatase | 0.70 | <0.001 |
| PTX3 | E2QZN8 | Pentraxin 3 | 2.57 | 0.0019 |
| QSOX1 | A0A5F4CZM1 | Sulfhydryl oxidase | 0.96 | <0.001 |
| RARRES2 | A0A5F4DC08 | Chemerin | 1.23 | 0.0350 |
| RBP4 | A0A5F4CED7 | Plasma retinol-binding protein | 0.49 | 0.0376 |
| RELN | F1PYH1 | Reelin | 1.12 | <0.001 |
| SERPINA3 | F1PH87 | SERPIN domain-containing protein | 0.34 | 0.0350 |
| SERPINA5 | E2RMF9 | SERPIN domain-containing protein | 0.99 | 0.0011 |
| SERPINA7 | A0A5F4D9Z3 | Serpin family A member 7 | 1.04 | 0.0012 |
| SERPINC1 | E2RES2 | Antithrombin-III | 0.36 | 0.0253 |
| SERPIND1 | E2RLN1 | Serpin family D member 1 | 0.29 | 0.0274 |
| SERPINF1 | A0A5F4CK29 | Serpin family F member 1 | 0.41 | 0.0098 |
| SMPDL3A | J9NYQ3 | Acid sphingomyelinase-like | 0.90 | <0.001 |
| | | phosphodiesterase | | |
| SPP2 | F1PD34 | Secreted phosphoprotein 2 | 1.23 | 0.0027 |
| TF | E2QZQ1 | Transferrin | 0.27 | 0.0160 |
| URAH | E2R5E9 | 5-hydroxyisourate hydrolase | 1.48 | 0.0012 |
| VCAM1 | F1PCD0 | Vascular cell adhesion protein 1 | 0.59 | <0.001 |
| VSIG4 | A0A5F4CA66 | V-set and immunoglobulin domain | 1.77 | <0.001 |
| | | containing 4 | | |
| VTN | A0A5F4C7K2 | Vitronectin | 0.35 | 0.0367 |
| VWF | A0A5F4C6P6 | von Willebrand factor | 0.92 | <0.001 |

TABLE 8
Molecules significantly downregulated in serum
from MPS I animals compared to controls.

| Symbol | Accession | Full Name | Log2 FC | p value |
|---|---|---|---|---|
| ACADM | F1P6W6 | Medium-chain specific acyl-CoA | −0.70 | 0.011 |
| | | dehydrogenase, mitochondrial | | |
| AFP | F2Z4Q6 | Alpha fetoprotein | −1.28 | 0.026 |
| ALB | P49822 | Albumin | −0.83 | <0.001 |
| ANPEP | A0A5F4CIA0 | Aminopeptidase | −0.80 | 0.011 |
| ANTXR1 | A0A5F4BVB4 | ANTXR cell adhesion molecule 1 | −0.95 | 0.017 |
| ANTXR2 | A0A5F4BTK2 | Anthrax toxin receptor | −1.40 | 0.022 |
| AOC3 | A0A5F4C6Q3 | Amine oxidase | −1.36 | <0.001 |
| APOC1 | P56595 | Apolipoprotein C-I | −0.50 | 0.026 |
| APOC2 | P12278 | Apolipoprotein C-II | −0.64 | 0.020 |
| APOC3 | A0A5F4DFV1 | Apolipoprotein C-III | −2.45 | 0.037 |
| APOC4 | E2RGB0 | Apolipoprotein C-IV | −1.38 | 0.005 |
| APOD | A0A5F4C2D5 | Apolipoprotein D | −0.47 | 0.001 |
| CA2 | A0A5F4CJB2 | Carbonic anhydrase | −0.75 | 0.014 |
| CAMP | A0A5F4CFZ9 | Cathelicidin antimicrobial peptide | −1.57 | 0.001 |
| CD5L | F1PG16 | CD5 molecule like | −0.50 | 0.010 |
| CEL | A0A5F4CJH2 | Carboxyl ester lipase | −1.35 | 0.004 |
| CFP | E2RH18 | Complement factor properdin | −1.10 | 0.019 |
| CLEC3B | E2RPB8 | C-type lectin domain family 3 member B | −0.75 | 0.004 |
| CLTA | J9NX46 | Clathrin light chain A | −2.17 | 0.002 |
| COL6A1 | F1PFM6 | Collagen type VI alpha 1 chain | −1.01 | 0.002 |
| COMP | A0A5F4CAI9 | Cartilage oligomeric matrix protein | −1.16 | 0.009 |
| CTSS | F1PAK0 | Cathepsin S | −0.67 | 0.030 |
| EIF4A2 | A0A0B4J198 | Eukaryotic translation initiation factor 4A2 | −1.08 | <0.001 |
| EXTL2 | F1PCE2 | Exostosin like glycosyltransferase 2 | −0.33 | 0.028 |
| FLT4 | A0A5F4C6I1 | Vascular endothelial growth factor receptor | −0.61 | 0.001 |
| | | 3 | | |
| HBB | A0A5F4D7C9 | hemoglobin subunit beta-like | −0.73 | 0.012 |
| IGKC | F1PNY2 | Immunoglobulin kappa constant | −0.83 | 0.003 |
| IGLL5 | A0A5F4BVD1 | Likely Immunoglobulin lambda-like | −0.74 | 0.008 |
| | | polypeptide 5 | | |
| ISLR | E2RM78 | Immunoglobulin superfamily containing | −1.11 | 0.005 |
| | | leucine rich repeat | | |
| ITGA2 | A0A5F4BUK5 | Integrin subunit alpha 2 | −1.39 | 0.015 |
| KCTD12 | E2RJY0 | Potassium channel tetramerization domain | −0.88 | 0.007 |
| | | containing 12 | | |
| LAMB1 | A0A5F4C4G4 | Laminin subunit beta 1 | −0.37 | 0.030 |
| LUM | E2R416 | Lumican | −0.60 | 0.010 |
| MGAM | E2RT38 | Maltase-glucoamylase | −1.07 | <0.001 |
| N/A | A0A5F4CWK7 | Ig-like domain-containing protein | −0.58 | 0.022 |
| N/A | E2RCC8 | Uncharacterized protein | −0.58 | 0.005 |
| N/A | A0A5F4DJC7 | Ig-like domain-containing protein | −0.66 | 0.003 |
| N/A | A0A5F4CLB9 | Ig-like domain-containing protein | −0.83 | 0.022 |
| N/A | A0A5F4CSF0 | Ig-like domain-containing protein | −0.90 | 0.019 |
| N/A | J9P6X8 | Uncharacterized protein | −1.06 | 0.010 |
| N/A | P01784 | Ig heavy chain V region GOM | −1.06 | <0.001 |
| N/A | A0A5F4BW09 | Ig-like domain-containing protein | −1.08 | 0.006 |
| N/A | A0A5F4BR98 | Ig-like domain-containing protein | −1.09 | 0.013 |
| N/A | A0A5F4BV52 | Ig-like domain-containing protein | −1.10 | 0.004 |
| N/A | L7N0E8 | Ig-like domain-containing protein | −1.12 | 0.010 |
| N/A | P01785 | Ig heavy chain V region MOO | −1.24 | <0.001 |
| N/A | A0A5F4CCT7 | IGv domain-containing protein | −1.39 | 0.006 |
| N/A | A0A5F4DCG3 | Ig-like domain-containing protein | −1.52 | 0.012 |
| N/A | A0A5F4CSD5 | Ig-like domain-containing protein | −1.55 | 0.003 |
| N/A | A0A5F4CBH6 | Ig-like domain-containing protein | −1.58 | <0.001 |
| N/A | F1PKL6 | Ig-like domain-containing protein | −1.81 | 0.011 |
| N/A | J9P0D4 | Ig-like domain-containing protein | −1.97 | 0.008 |
| N/A | A0A5F4DAX5 | Ig-like domain-containing protein | −1.98 | 0.002 |
| N/A | A0A5F4BUM4 | Ig-like domain-containing protein | −1.99 | 0.006 |
| N/A | J9NY02 | Ig-like domain-containing protein | −2.28 | 0.011 |
| N/A | F1PH61 | Ig-like domain-containing protein | −2.41 | 0.035 |
| N/A | A0A5F4C262 | Ig-like domain-containing protein | −2.61 | <0.001 |
| N/A | A0A5F4BTN9 | Ig-like domain-containing protein | −4.61 | 0.013 |
| PEPD | F1PNS6 | Peptidase D | −0.74 | 0.000 |
| PLXDC1 | A0A5F4BY14 | Plexin domain containing 1 | −0.73 | 0.016 |
| PON3 | E2RPW3 | Paraoxonase 3 | −0.21 | 0.031 |
| PYGL | A0A5F4CFX8 | Alpha-1,4 glucan phosphorylase | −2.23 | 0.025 |
| SLC3A2 | F1PRC5 | Solute carrier family 3 member 2 | −0.93 | 0.027 |
| SOD1 | Q8WNN6 | Superoxide dismutase | −1.03 | 0.029 |
| TFRC | F1PEN6 | Transferrin receptor protein 1 | −0.91 | 0.004 |

### References

[1] J. Muenzer, Overview of the mucopolysaccharidoses, Rheumatology (Oxford) 50 Suppl 5 (2011) pp v4-12;
[2] C. S. Hampe, J. B. Eisengart, T. C. Lund, P. J. Orchard, M. Swietlicka, J. Wesley, R. S. McIvor, Mucopolysaccharidosis Type I: A Review of the Natural History and Molecular Pathology, Cells 9(8) (2020) pp 1-26;
[3] C. M. Simonaro, M. D'Angelo, X. He, E. Eliyahu, N. Shtraizent, M. E. Haskins, E. H. Schuchman, Mechanism of glycosaminoglycan-mediated bone and joint disease: implications for the mucopolysaccharidoses and other connective tissue diseases, Am J Pathol 172(1) (2008) pp 112-22;
[4] S. H. Peck, P. J. O'Donnell, J. L. Kang, N. R. Malhotra, G. R. Dodge, M. Pacifici, E. M. Shore, M. E. Haskins, L. J. Smith, Delayed hypertrophic differentiation of epiphyseal chondrocytes contributes to failed secondary ossification in mucopolysaccharidosis VII dogs, Mol Genet Metab 116(3) (2015) pp 195-203;
[5] J. O. Stracke, A. J. Fosang, K. Last, F. A. Mercuri, A. M. Pendas, E. Llano, R. Perris, P. E. Di Cesare, G. Murphy, V. Knauper, Matrix metalloproteinases 19 and 20 cleave aggrecan and cartilage oligomeric matrix protein (COMP), FEBS Lett 478(1-2) (2000) pp 52-6;
[6] Y. Miyake, K. Tanaka, M. Arakawa, ITIH3 and ITIH4 polymorphisms and depressive symptoms during pregnancy in Japan: the Kyushu Okinawa Maternal and Child Health Study, J Neural Transm (Vienna) 125(10) (2018) pp 1503-1509;
[7] J. Ma, D. S. Niu, N. J. Wan, Y. Qin, C. J. Guo, Elevated chemerin levels in synovial fluid and synovial membrane from patients with knee osteoarthritis, Int J Clin Exp Pathol 8(10) (2015) pp 13393-8;
[8] D. M. Mohammed Ali, S. Z. Al-Fadhel, N. H. A. Al-Ghuraibawi, H. K. Al-Hakeim, Serum chemerin and visfatin levels and their ratio as possible diagnostic parameters of rheumatoid arthritis, Reumatologia 58(2) (2020) pp 67-75.

1. A method comprising:
(a) measuring a level of at least one protein in a sample from a subject, the at least one protein selected form the panel of proteins as presented in Table 2, wherein the level of the at least one protein has a log 2 fold change between about −2.5 and about 5 relative to the level of the at least one protein in a sample from a control subject, and
(b) administering to the subject a mucopolysaccharidoses (MPS) treatment selected from the group consisting of enzyme replacement therapy, bone marrow transplantation, surgery, and physical therapy.

2. The method of claim 1, comprising measuring the level of at least two proteins selected from the panel.

3. The method of claim 1, comprising measuring the level of at least three proteins selected from the panel.

4. The method of claim 1, comprising measuring the level of at least four proteins selected from the panel.

5. The method of claim 1, comprising measuring the level of at least five proteins selected from the panel.

6. The method of claim 1, comprising measuring the level of at least six proteins selected from the panel.

7. The method of claim 1, comprising measuring the level of at least seven proteins selected from the panel.

8. The method of claim 1, comprising measuring the level of at least eight proteins selected from the panel.

9. The method of claim 1, comprising measuring the level of at least nine proteins selected from the panel.

10. The method of claim 1, comprising measuring the level of at least ten proteins selected from the panel.

11. The method of claim 1, wherein the at least one protein is selected from matrix metalloproteinase 19 (MMP19), inter-alpha-trypsin inhibitor heavy chain 3 (ITIH3), chemerin (RARRES2), C-reactive protein (CRP), alpha-mannosidase (MAN2B1), cluster differentiation (CD)86, alpha-1-microglobulin (AMBP), prosaposin (PSAP), secreted phosphoprotein 2 (SPP2), and V-set and immunoglobulin domain containing 4 (VSIG4).

12. The method of claim 1, wherein administering the treatment to the subject occurs earlier in the life of the subject with step (a) than without step (a).

13. The method of claim 1, wherein the sample is selected from the group consisting of synovial fluid, blood, serum, saliva, urine, and pleural fluid.

14. The method of claim 13, wherein the sample is synovial fluid treated with hyaluronidase.

15. The method of claim 1, wherein measuring comprises measuring by a method selected from the group consisting of Western blotting, enzyme-linked immunosorbent assay (ELISA), multiplex ELISA, and high performance liquid chromatography (HPLC).

16. The method of claim 1, wherein the subject is a human.

17. The method of claim 1, wherein the subject is a child.

18. The method of claim 1, wherein the subject is an adolescent.

19. The method of claim 1, wherein the subject has diminished biomechanical properties of anterior cruciate ligament (ACL).

20. The method of claim 1, wherein the level of the at least one protein has between about 0.2 and about 30 fold change relative to the level of the at least one protein in the sample from the control subject.

21. A method comprising:
(a) measuring a level of between one and ten proteins in a sample from a subject, wherein the protein(s) are selected form the panel of proteins as presented in Table 2, and wherein the level of the protein(s) has a log 2 fold change between about −2.5 and about 5 relative to the level of the protein(s) in a sample from a control subject, and
(b) administering to the subject a mucopolysaccharidoses (MPS) treatment, wherein the administering is earlier in the life of the subject with step (a) than without step (a).

22. The method of claim 21, wherein the protein(s) are selected from matrix metalloproteinase 19 (MMP19), inter-alpha-trypsin inhibitor heavy chain 3 (ITIH3), chemerin (RARRES2), C-reactive protein (CRP), alpha-mannosidase (MAN2B1), cluster differentiation (CD)86, alpha-1-microglobulin (AMBP), prosaposin (PSAP), secreted phosphoprotein 2 (SPP2), and V-set and immunoglobulin domain containing 4 (VSIG4).

23. The method of claim 21, wherein the subject has diminished biomechanical properties of anterior cruciate ligament (ACL).

24. The method of claim 1, wherein the level of the at least one protein has between about 0.2 and about 30 fold change relative to the level of the at least one protein in the sample from the control subject.

25. The method of claim 1, wherein the control subject is a healthy subject.

26. The method of claim 1, wherein the control subject is a subject who does not suffer from mucopolysaccharidoses (MPS).
